(19) United States
(12) Patent Application Publication
(10) Pub. No.: US 2025/0215126 A1
(43) Pub. Date: Jul. 3, 2025

(54) COMPOSITION, SUBSTRATE TREATMENT METHOD, MANUFACTURING METHOD OF SEMICONDUCTOR DEVICE, AND COMPOUND

(71) Applicant: FUJIFILM CORPORATION, Tokyo (JP)

(72) Inventors: Naoya Shimoju, Shizuoka (JP); Yasuo Sugishima, Shizuoka (JP); Tomonori Takahashi, Shizuoka (JP)

(21) Appl. No.: 19/081,442

(22) Filed: Mar. 17, 2025

Related U.S. Application Data
(63) Continuation of application No. PCT/JP2023/034494, filed on Sep. 22, 2023.

(30) Foreign Application Priority Data
Sep. 30, 2022 (JP) 2022-158521

(51) Int. Cl. C08F 212/08; C08F 222/02; C11D 1/00

(52) U.S. Cl. CPC C08F 212/08; C08F 222/02; C11D 1/008; C11D 2111/22

(57) ABSTRACT

The present invention provides a composition for a semiconductor device, which has excellent removability of dry etching residues, further suppresses dissolution of tungsten, and is unlikely to leave residues after being applied to an object to be treated and then subjected to a rinsing treatment. The composition for a semiconductor device of the present invention contains a resin including a repeating unit A which has a specific group selected from the group consisting of a primary amino group, a secondary amino group, a tertiary amino group, and salts of these groups, and has a hydroxy group, and a repeating unit B which has a functional group having a pKa of 10.0 or less or a salt of the functional group, and contains water.

### CROSS-REFERENCE TO RELATED APPLICATIONS

This application is a Continuation of PCT International Application No. PCT/JP2023/034494 filed on Sep. 22, 2023, which claims priority under 35 U.S.C. § 119(a) to Japanese Patent Application No. 2022-158521 filed on Sep. 30, 2022. The above applications are hereby expressly incorporated by reference, in their entirety, into the present application.

### BACKGROUND OF THE INVENTION

### 1. Field of the Invention

The present invention relates to a composition, a substrate treatment method, a manufacturing method of a semiconductor device, and a compound.

### 2. Description of the Related Art

With the progress of miniaturization of semiconductor products, there is an increasing demand for performing a step of removing unnecessary metal-containing substances on a substrate with high efficiency and accuracy in a semiconductor product manufacturing process.

Semiconductor devices are manufactured, for example, by disposing, on a substrate, a laminate having a metal layer serving as a wiring line material, an etching stop film, and an insulating film, forming a resist film on this laminate, and performing a photolithography step and a dry etching step.

In the photolithography step, a method of carrying out etching or a method of removing foreign substances which have adhered to the surface of the substrate, using a composition which dissolves the metal-containing substance, has been widely known.

For example, in the photolithography step, the metal layer and/or the insulating film on the substrate may be etched by a dry etching treatment using a resist film as a mask. In this case, residues and the like derived from the metal layer and/or the insulating film may adhere to the substrate, the metal layer, and the insulating film. In order to remove the adhered residues, washing using a composition is usually carried out.

In addition, the resist film which is used as a mask in the etching is removed from the laminate by a dry-type method (dry ashing) with ashing (incineration) or a wet-type method. The residues derived from the resist film and the like may adhere to the laminate from which the resist has been removed by the dry ashing method.

Furthermore, nowadays, a resist film (so-called metal hard mask) based on a metal material such as TiN and AlOx has been studied as the resist film in order to achieve further miniaturization of semiconductor devices. In a case where the metal hard mask is used as the resist film, a dry etching step (for example, a plasma etching treatment) is usually performed using the metal hard mask as a mask, and a step of forming holes based on a pattern shape of the metal hard mask and exposing a surface of a metal film serving as a wiring line film are usually performed.

Etching residues and/or ashing residues are deposited on the substrate which has undergone the dry etching step or the dry ashing step. In a case where the metal hard mask is used as the resist film, a residue component contains, for example, a large amount of a metal component such as a titanium-based metal; and in a case where a photoresist film is used as the resist film, a residue component contains a large amount of an organic component.

In order to prevent these adhered residues from interfering with the subsequent step, a washing treatment of removing the residues is usually carried out using a composition. In addition, examples of the wet-type method of removing the resist film include an aspect in which the resist film is removed using a composition.

The composition for a semiconductor device as described above is used for a treatment such as the removal of metal-containing substances (etching residues and ashing residues) on the substrate and/or the removal of the resist film in the manufacturing process of the semiconductor device.

For example, JP2012-094852A discloses a washing solution which is used in a washing step for a substrate for a semiconductor device, in which the washing solution is a washing solution for a substrate for a semiconductor device, containing at least one polymer coagulating agent selected from an organic acid, a sulfonic acid-type anionic surfactant, polyvinylpyrrolidone, or a polyethylene oxide-polypropylene oxide block copolymer, and water.

### SUMMARY OF THE INVENTION

As a result of using the washing solution containing polyvinylpyrrolidone as a composition for removing etching residues with respect to an object to be treated having tungsten (W) after a dry etching treatment, with reference to JP2012-094852A, the present inventors found that removability of the dry etching residues and performance of suppressing dissolution of tungsten are insufficient.

Furthermore, after washing the object to be treated with the composition as described above and then carrying out a rinsing treatment, residues are likely to remain.

An object of the present invention is to provide a composition for a semiconductor device, which has excellent removability of dry etching residues, further suppresses dissolution of tungsten, and is unlikely to leave residues after being applied to an object to be treated and then subjected to a rinsing treatment.

Another object of the present invention is to provide a substrate treatment method using the above-described composition, a manufacturing method of a semiconductor device, and a compound.

The present inventors have completed the present invention as a result of intensive studies to solve the above-described problems. That is, the present inventors have found that the above-described objects can be achieved by the following configuration.

[1] A composition for a semiconductor device, comprising:
a resin including a repeating unit A which has a specific group selected from the group consisting of a primary amino group, a secondary amino group, a tertiary amino group, and salts of these groups, and has a hydroxy group, and
   a repeating unit B which has a functional group having a pKa of 10.0 or less or a salt of the functional group; and water.

[2] The composition for a semiconductor device according to [1],
in which the repeating unit A is a repeating unit derived from a compound represented by Formula (A) described later, a repeating unit derived from a compound represented by Formula (B) described later, or a repeating unit represented by Formula (C) described later.

[3] The composition for a semiconductor device according to [1] or [2],
in which at least one of the specific group is a primary amino group or a salt of the primary amino group.

[4] The composition for a semiconductor device according to [2] or [3],
in which the repeating unit A is the repeating unit derived from the compound represented by Formula (A), in which L^{a} is an (na+2)-valent linking group including an aromatic ring, the repeating unit derived from the compound represented by Formula (B), in which at least one of L^{b1} or L^{b2} is a divalent linking group including an aromatic ring, or the repeating unit represented by Formula (C).

[5] The composition for a semiconductor device according to [1] or [3],
in which the repeating unit A is a repeating unit derived from a compound represented by Formula (D) described later or a repeating unit represented by Formula (E) described later.

[6] The composition for a semiconductor device according to any one of [1] to [5],
in which the pKa of the functional group is 5.0 or less.

[7] The composition for a semiconductor device according to any one of [1] to [6],
in which the functional group is a carboxy group.

[8] The composition according to any one of [1] to [7], further comprising:
at least one selected from the group consisting of a removing agent, an oxidizing agent, a corrosion inhibitor, a surfactant, an antifoaming agent, and an organic solvent.

[9] The composition according to any one of [1] to [8], further comprising:
a removing agent.

[10] A substrate treatment method comprising:
a step A of bringing the composition according to any one of [1] to [9] into contact with a substrate having a metal-containing substance.

[11] The substrate treatment method according to [10], further comprising, after the step A:
a step B of subjecting the substrate obtained in the step A to a rinsing treatment using a rinsing liquid.

[12] A manufacturing method of a semiconductor device, comprising:
the substrate treatment method according to [10] or [11].

[13] A compound represented by Formula (F) described later.

According to the present invention, it is possible to provide a composition for a semiconductor device, which has excellent removability of dry etching residues, further suppresses dissolution of tungsten, and is unlikely to leave residues after being applied to an object to be treated and then subjected to a rinsing treatment.

In addition, according to the present invention, it is possible to provide a substrate treatment method using the above-described composition, a manufacturing method of a semiconductor device, and a compound.

### BRIEF DESCRIPTION OF THE DRAWINGS

FIG. 1 is a schematic cross-sectional view showing an example of a laminate which is an object to be treated in a substrate treatment method.

### DESCRIPTION OF THE PREFERRED EMBODIMENTS

Hereinafter, the present invention will be described in detail.

The description of the configuration requirements described below is made on the basis of representative embodiments of the present invention, but it should not be construed that the present invention is limited to those embodiments.

Hereinafter, meaning of each description in the present specification will be explained.

In the present specification, the numerical value range indicated by “to” means a range including numerical values before and after “to” as a lower limit value and an upper limit value, respectively.

In the present specification, in a case where two or more kinds of certain components are present, “content” of the component means a total content of the two or more kinds of the components.

In the present specification, “total solid content” means the total content of all components contained in a composition, other than a solvent such as water and an organic solvent.

In the present specification, “preparing” includes not only preparing a predetermined substance by a treatment such as synthesis and blending of raw materials, but also procuring a predetermined substance by purchasing or the like.

A compound described in the present specification may include a structural isomer, an optical isomer, and an isotope unless otherwise specified. In addition, one kind of structural isomer, optical isomer, and isotope alone, or two or more kinds thereof may be included.

In the present specification, unless otherwise specified, a bonding direction of a divalent group (for example, —COO—) is not limited. For example, in a case where Y in a compound represented by a formula “X—Y—Z” is-COO—, the compound may be “X—O—CO—Z” or “X—CO—O—Z”.

In the present specification, unless otherwise specified, a symbol “*” specified in the chemical formula represents a bonding position.

In the present specification, “ppm” means “parts-per-million (10⁻⁶)”, “ppb” means “parts-per-billion (10⁻⁹)”, and “ppt” means “parts-per-trillion (10⁻¹²)”.

In the present specification, 1 angstrom (Å) corresponds to 0.1 nm.

In the present specification, unless otherwise specified, the weight-average molecular weight (Mw) and the number average molecular weight (Mn) are values obtained using TSKgel GMHxL, TSKgel G4000HxL, or TSKgel G2000HxL (all of which are manufactured by Tosoh Corporation) as a column, using tetrahydrofuran as an eluent, using a differential refractometer as a detector, using polystyrene as a standard substance, and carrying out conversion using the polystyrene as a standard substance, which has been subjected to measurement with a gel permeation chromatography (GPC) analysis apparatus.

In the present specification, unless otherwise specified, a molecular weight of a compound having a molecular weight distribution is a weight-average molecular weight.

### [Composition]

Hereinafter, the composition according to the embodiment of the present invention (hereinafter, also referred to as “present composition”) will be described in detail.

The present composition is a composition for a semiconductor device, that contains a resin (hereinafter, also referred to as “specific resin”) including a repeating unit A which has a specific group selected from the group consisting of a primary amino group, a secondary amino group, a tertiary amino group, and salts of these groups, and has a hydroxy group, and a repeating unit B which has a functional group having a pKa of 10.0 or less or a salt of the functional group, and water.

The present inventors have found that, by using the present composition containing the above-described specific resin and water, dissolution of tungsten is further suppressed, removability of residues is excellent, and an amount of residues is small even after a rinsing treatment, and have completed the present invention. Hereinafter, the fact that at least one of that the dissolution of tungsten is further suppressed, that the removability of residues is excellent, or that the amount of residues is small even after a rinsing treatment is more excellent is also referred to as “effect of the present invention is more excellent”.

Although the mechanism by which the object of the present invention can be achieved by adopting the above-described configuration of the present composition is not necessarily clear, the present inventors presume as follows.

The mechanism by which the object can be achieved is not limited by the following supposition. In other words, even in a case where the object can be achieved by a mechanism other than the following, it is included in the scope of the present invention.

In a case where the present composition is used for treating an object to be treated, containing tungsten, since the above-described repeating unit A has both the hydroxy group which is easily adsorbed on the tungsten, and the specific group, it is easy to form a suitable protective film composed of the specific resin on a surface of the tungsten.

Here, in a case of a resin having only the specific group, such as polyethyleneimine, or a resin in which the hydroxy group and the specific group are present in repeating units different from each other, a sufficient effect cannot be obtained, whereas in a case of a resin in which the hydroxy group and the specific group are present in the same repeating unit, such as the specific resin, the dissolution of tungsten can be suppressed.

Although the detailed mechanism is not clear, the present inventors presume that the hydroxy group and the specific group are in a positional relationship close to each other, and as a result, the above-described protective film is more effectively formed, and thus the dissolution of tungsten can be further suppressed.

In addition, the present composition includes the above-described repeating unit B, and thus has more excellent affinity with a rinsing liquid. Therefore, the removability of residues (for example, residues derived from the resin in the present composition) in a case where the present composition is applied to the object to be treated and then subjected to a rinsing treatment is also excellent.

Hereinafter, each component contained in the present composition will be described in detail.

### [Specific Resin]

The present composition contains a resin (specific resin) including the repeating unit A and the repeating unit B.

### <Repeating Unit A>

The above-described repeating unit A is a repeating unit having a specific group selected from the group consisting of a primary amino group, a secondary amino group, a tertiary amino group, and salts thereof, and having a hydroxy group.

The primary amino group refers to a group represented by —NH₂; the secondary amino group refers to a group represented by —NHR^{A1} (R^{A1} represents a monovalent aliphatic hydrocarbon group or a monovalent aromatic ring group); and the tertiary amino group refers to a group represented by —N(R^{A2})₂ (R^{A2}'s each independently represent a monovalent aliphatic hydrocarbon group or a monovalent aromatic ring group).

The monovalent aliphatic hydrocarbon group represented by R^{A1} or R^{A2} may be linear, branched, or cyclic.

The aliphatic hydrocarbon group may further have a substituent. In a case where the aliphatic hydrocarbon group further has a substituent, it is preferable that the aliphatic hydrocarbon group has the specific group, a hydroxy group, or a halogen atom.

The number of carbon atoms in the aliphatic hydrocarbon group is preferably 1 to 10, more preferably 1 to 6, and still more preferably 1 or 2.

Examples of the aliphatic hydrocarbon group include an alkyl group, an alkenyl group, and an alkynyl group; and among these, an alkyl group having 1 to 6 carbon atoms is preferable, and an alkyl group having 1 or 2 carbon atoms is more preferable.

An aromatic ring constituting the monovalent aromatic ring group represented by R^{A1} or R^{A2} may be monocyclic or polycyclic.

The number of ring member atoms in the aromatic ring constituting the monovalent aromatic ring group is preferably 5 to 20, more preferably 5 to 10, and still more preferably 5 or 6.

In addition, the above-described monovalent aromatic ring group may be an aryl group or a heteroaryl group.

As a heteroatom included in the heteroaryl group, an oxygen atom, a nitrogen atom, or a sulfur atom is preferable.

The above-described salt is a salt containing at least one group (specific group) selected from the group consisting of the primary amino group, the secondary amino group, and the tertiary amino group.

A compound which forms the salt is not particularly limited, and examples thereof include an acidic compound. That is, the above-described salt is preferably a salt formed of at least one group selected from the group consisting of the primary amino group, the secondary amino group, and the tertiary amino group, and an acidic compound.

The acidic compound may be an inorganic acid or an organic acid.

Examples of the inorganic acid include hydrochloric acid, sulfuric acid, phosphoric acid, and nitric acid. Examples of the organic acid include acetic acid, propionic acid, methanesulfonic acid, ethanesulfonic acid, allylglycine, maleic acid, citraconic acid, fumaric acid, and itaconic acid.

As the acid in a case where the primary amino group, the secondary amino group, or the tertiary amino group forms the salt, hydrochloric acid, acetic acid, propionic acid, methanesulfonic acid, or ethanesulfonic acid is preferable, and hydrochloric acid, acetic acid, or ethanesulfonic acid is more preferable.

The number of specific groups in the above-described repeating unit A is not particularly limited, but is preferably 1 to 5, more preferably 1 or 2, and still more preferably 1.

In a case where the repeating unit A includes a plurality of the above-described specific groups, it is preferable that at least one of the above-described specific groups is a primary amino group or a salt thereof.

In addition, the number of hydroxy groups in the repeating unit A is not particularly limited, but is preferably 1 to 5, more preferably 1 or 2, and still more preferably 1.

The hydroxy group in the repeating unit A is preferably a hydroxy group bonded to a carbon atom in an aliphatic hydrocarbon (a hydroxy group which is not a phenolic hydroxy group).

It is also preferable that the repeating unit A includes an aromatic ring.

From the viewpoint that the effect of the present invention is more excellent, the above-described repeating unit A is preferably a repeating unit derived from a compound represented by Formula (A), a repeating unit derived from a compound represented by Formula (B), or a repeating unit represented by Formula (C).

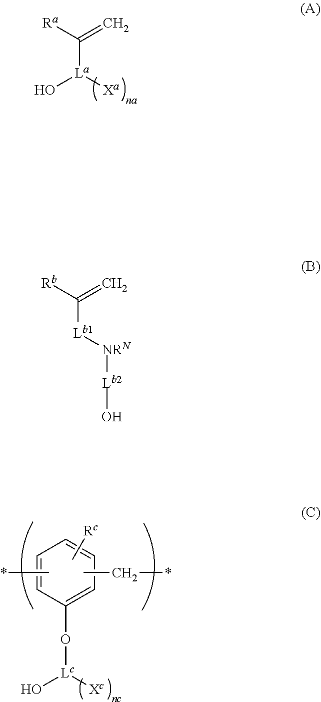

In Formula (A), R^{a} represents a hydrogen atom or an alkyl group having 1 to 4 carbon atoms.

Examples of the alkyl group having 1 to 4 carbon atoms, represented by R^{a}, include a methyl group, an ethyl group, an isopropyl group, and a t-butyl group.

R^{a} is preferably a hydrogen atom or a methyl group.

In Formula (A), X^{f} represents a specific group selected from the group consisting of a primary amino group, a secondary amino group, a tertiary amino group, and salts thereof.

Specific aspects and suitable aspects of X^{a} are as described for the specific group above.

In Formula (A), L^{a} represents an (na+2)-valent linking group, and na represents an integer of 1 to 5.

na is preferably 1 to 3, more preferably 1 or 2, and still more preferably 1.

Examples of the (na+2)-valent linking group represented by L^{a} include an (na+2)-valent aliphatic hydrocarbon group, an (na+2)-valent aromatic ring group, —O—, —CO—(carbonyl group), —SO₂—, —N<, —NR^{L}—(R^{L} represents a hydrogen atom or a monovalent aliphatic hydrocarbon group), and a group formed by a combination of these groups.

The above-described (na+2)-valent aliphatic hydrocarbon group is a group obtained by removing (na+2) hydrogen atoms from an aliphatic hydrocarbon.

The above-described aliphatic hydrocarbon may be linear, branched, or cyclic, but is preferably linear.

In addition, the number of carbon atoms in the aliphatic hydrocarbon is preferably 1 to 6, more preferably 1 to 3, and still more preferably 1 or 2.

Examples of the aliphatic hydrocarbon include an alkane, an alkene, and an alkyne.

The above-described (na+2)-valent aromatic ring group is a group obtained by removing (na+2) hydrogen atoms from an aromatic hydrocarbon ring or an aromatic heterocyclic ring.

The aromatic hydrocarbon ring and the aromatic heterocyclic ring may be monocyclic or polycyclic.

The number of ring member atoms in the aromatic ring constituting the monovalent aromatic ring group is preferably 5 to 20, more preferably 5 to 10, and still more preferably 5 or 6.

Examples of the above-described aromatic ring include a benzene ring.

Examples of the above-described aromatic hydrocarbon ring include a benzene ring, a naphthalene ring, an anthracene ring, a fluorene ring, and a phenanthrene ring.

Examples of the above-described aromatic heterocyclic ring include a pyridine ring, a pyrimidine ring, a pyrrole ring, a furan ring, a thiophene ring, an imidazole ring, an oxazole ring, a thiazole ring, a benzofuran ring, a benzothiophene ring, a benzimidazole ring, a benzoxazole ring, a benzothiazole ring, and a benzothiadiazole ring.

Examples of the monovalent aliphatic hydrocarbon group represented by R^{L} in the group represented by —NR^{L}-described above include an alkyl group having 1 to 10 carbon atoms.

Among these, R^{L} is preferably a hydrogen atom or an alkyl group having 1 to 6 carbon atoms, and more preferably a hydrogen atom or an alkyl group having 1 or 2 carbon atoms.

L^{a} is also preferably an (na+2)-valent linking group including an aromatic ring.

The above-described aromatic ring may be an aromatic hydrocarbon ring or an aromatic heterocyclic ring.

Examples of the above-described aromatic hydrocarbon ring include a benzene ring and a naphthalene ring, and a benzene ring is preferable.

Examples of the above-described aromatic heterocyclic ring include an imidazole ring, a pyrazole ring, a thiazole ring, a triazole ring, a tetrazole ring, a pyridine ring, a pyrazine ring, a pyrimidine ring, and a triazine ring; and among these rings, an imidazole ring, a pyridine ring, or a triazine ring is preferable.

Examples of the (na+2)-valent linking group represented by L^{a} in a case where na is 1 include *-divalent aromatic ring group-O-trivalent aliphatic hydrocarbon group<, *—CO—O-trivalent aliphatic hydrocarbon group<, a trivalent aromatic ring group, *—CO—NH-divalent aromatic ring group-O-trivalent aliphatic hydrocarbon group<, *—O-trivalent aliphatic hydrocarbon group<, *-divalent aromatic ring group-CO—O-trivalent aliphatic hydrocarbon group<, *-divalent aromatic ring group-divalent aliphatic hydrocarbon group-O-trivalent aliphatic hydrocarbon group<, and *—CO—O-trivalent aliphatic hydrocarbon group-NH-divalent aliphatic hydrocarbon-.

* represents a bonding position to the carbon atom to which R^{a} in Formula (A) is bonded.

The above-described group represented by *—CO—O-trivalent aliphatic hydrocarbon group-NH-divalent aliphatic hydrocarbon group- is a group represented by Formula (L), in which R^{al3} represents a trivalent aliphatic hydrocarbon group and R^{al2} represents a divalent aliphatic hydrocarbon group.

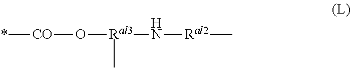

In Formula (B), R^{b} represents a hydrogen atom or an alkyl group having 1 to 4 carbon atoms.

Specific aspects and preferred aspects of the alkyl group having 1 to 4 carbon atoms, represented by R^{b}, are the same as the specific aspects and the preferred aspects of the above-described alkyl group having 1 to 4 carbon atoms, which may have a substituent, represented by R^{a}.

R^{b} is preferably a hydrogen atom or a methyl group.

In Formula (B), R^{N} represents a hydrogen atom or an alkyl group which may have a substituent.

The above-described alkyl group which may have a substituent may be linear, branched, or cyclic.

The number of carbon atoms in the alkyl group which may have a substituent is preferably 1 to 10, more preferably 1 to 6, and still more preferably 1 or 2.

In a case where the alkyl group represented by R^{N} further has a substituent, it is preferable that the alkyl group has the specific group or a hydroxy group.

In Formula (B), L^{b1} and L^{b2} each independently represent a single bond or a divalent linking group.

Examples of the divalent linking group represented by L^{b1} and L^{b2} include a divalent aliphatic hydrocarbon group, a divalent aromatic ring group, —O—, —CO—(carbonyl group), and a group formed by a combination of these groups.

The above-described divalent aliphatic hydrocarbon group may be linear, branched, or cyclic, but is preferably linear.

In addition, the number of carbon atoms in the divalent aliphatic hydrocarbon group is preferably 1 to 6, more preferably 1 to 3, and still more preferably 1 or 2.

Examples of the divalent aliphatic hydrocarbon group include an alkylene group and an alkenylene group.

The above-described divalent aromatic ring group may be an arylene group or a heteroarylene group, and an aromatic ring constituting the divalent aromatic ring group may be monocyclic or polycyclic.

The number of ring member atoms in the aromatic ring constituting the divalent aromatic ring group is preferably 5 to 20, more preferably 5 to 10, and still more preferably 5 or 6.

Examples of the above-described aromatic ring include a benzene ring.

Among these, L^{b1} and L^{b2} are preferably an alkylene group, more preferably an alkylene group having 1 to 6 carbon atoms, and still more preferably a methylene group or an ethylene group.

In addition, it is also preferable that at least one of L^{b1} or L^{b2} is a divalent linking group including an aromatic ring. Specific aspects and suitable aspects of the above-described aromatic ring are the same as the specific aspects and the suitable aspects of the aromatic ring which can be included in L^{a}.

In Formula (C), R^{c} represents a hydrogen atom or a methyl group.

X^{c} represents a specific group selected from the group consisting of a primary amino group, a secondary amino group, a tertiary amino group, and salts of these groups.

Specific aspects and suitable aspects of X^{c} are as described for the specific group above.

In Formula (C), L^{a} represents an (nc+2)-valent linking group, and nc represents an integer of 1 to 5.

nc is preferably 1 to 3, more preferably 1 or 2, and still more preferably 1.

Examples of the (nc+2)-valent linking group represented by L^{e} include an (nc+2)-valent aliphatic hydrocarbon group, an (nc+2)-valent aromatic ring group, —O—, —CO— (carbonyl group), —SO₂—, —N<, —NR^{L}—(R^{L} represents a hydrogen atom or a monovalent aliphatic hydrocarbon group), and a group formed by a combination of these groups.

Specific aspects and suitable aspects of these linking groups are as described in detail for the (na+2)-valent linking group represented by L^{a}.

Among these, the (nc+2)-valent linking group represented by L^{e} is preferably an (nc+2)-valent aliphatic hydrocarbon group.

Examples of the aliphatic hydrocarbon include an alkane, an alkene, and an alkyne.

In addition, the number of carbon atoms in the aliphatic hydrocarbon is preferably 1 to 10, more preferably 1 to 6, and still more preferably 1 to 3.

It is also preferable that the above-described repeating unit A is a repeating unit derived from a compound represented by Formula (D) or a repeating unit represented by Formula (E).

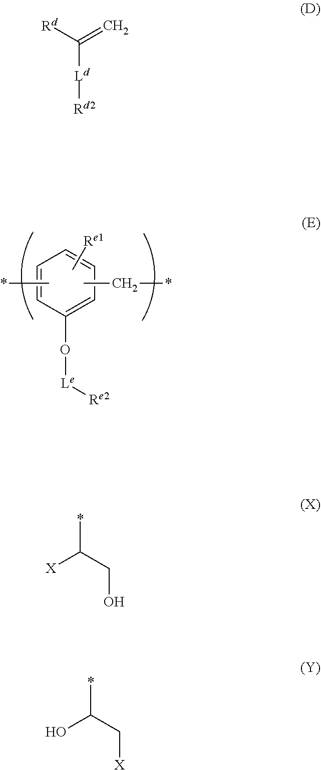

In Formula (D), R^{d} represents a hydrogen atom or an alkyl group having 1 to 4 carbon atoms.

Examples of the alkyl group having 1 to 4 carbon atoms, represented by R^{d}, include a methyl group, an ethyl group, and an isopropyl group.

In Formula (D), L^{d} represents a single bond or a divalent linking group.

Examples of the divalent linking group represented by L^{d} include a divalent aliphatic hydrocarbon group, a divalent aromatic ring group, —O—, —CO—(carbonyl group), —NR^{L}—(R^{L} represents a hydrogen atom or a monovalent aliphatic hydrocarbon group), and a group formed by a combination of these groups.

Specific aspects and suitable aspects of the divalent aliphatic hydrocarbon group and the divalent aromatic ring group described above are the same as the specific aspects and the suitable aspects of the divalent aliphatic hydrocarbon group and the divalent aromatic ring group represented by L^{b1} or L^{b2} in Formula (B).

Examples of the monovalent aliphatic hydrocarbon group represented by R^{L} in the group represented by —NR^{L}-described above include an alkyl group having 1 to 10 carbon atoms.

Among these, R^{L} is preferably a hydrogen atom or an alkyl group having 1 to 6 carbon atoms, and more preferably a hydrogen atom or an alkyl group having 1 or 2 carbon atoms.

Examples of the divalent linking group represented by L^{d} include-divalent aliphatic hydrocarbon group-, —COO—(ester bond)-divalent aliphatic hydrocarbon group-, —CONH— (amide bond)-divalent aliphatic hydrocarbon group-, -divalent aromatic ring group-O—, -divalent aromatic ring group-CH₂—O—, -divalent aromatic ring group-C≡C—CH₂—O—, and -divalent aromatic ring group-COO—.

In Formula (D), R^{d2} represents a group represented by Formula (X) or a group represented by Formula (Y).

In Formula (X) and Formula (Y), X represents a primary amino group or a salt thereof.

Specific aspects and suitable aspects of the salt of the primary amino group are as described above.

In Formula (E), R^{e1} represents a hydrogen atom or a methyl group.

In Formula (E), L^{e} represents a divalent linking group.

Examples of the divalent linking group represented by L^{e} include a divalent aliphatic hydrocarbon group, a divalent aromatic ring group, —O—, —CO—(carbonyl group), —NR^{L}— (R^{L} represents a hydrogen atom or a monovalent aliphatic hydrocarbon group), and a group formed by a combination of these groups.

Specific aspects and suitable aspects of the divalent aliphatic hydrocarbon group and the divalent aromatic ring group described above are the same as the specific aspects and the suitable aspects of the divalent aliphatic hydrocarbon group and the divalent aromatic ring group represented by L^{b1} or L^{b2} in Formula (B).

The divalent linking group represented by L^{e} is preferably a divalent aliphatic hydrocarbon group.

In Formula (E), R^{e2} represents the above-described group represented by Formula (X) or the above-described group represented by Formula (Y).

From the viewpoint that the effect of the present invention is more excellent, the above-described compound represented by Formula (D) is preferably a compound represented by Formula (F).

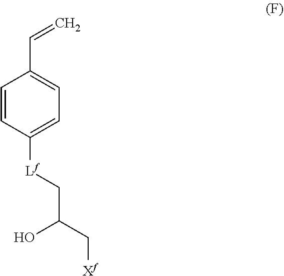

In Formula (F), X^{f} represents a specific group selected from the group consisting of a primary amino group, a secondary amino group, a tertiary amino group, and salts of these groups.

Specific aspects and suitable aspects of the primary amino group, the secondary amino group, the tertiary amino group, and salts thereof, which are represented by X^{f}, are the same as the specific aspects and the suitable aspects of X^{a} in Formula (A).

Among these, the above-described salt is preferably a hydrochloride, a sulfate, an acetate, or a carbonate.

L^{f} represents —CH₂O—, —COO—, —C≡C—CH₂O—, or —O—.

The specific resin may have only one kind of the above-described repeating unit A or may have two or more kinds thereof.

From the viewpoint that the effect of the invention is more excellent, a content of the repeating unit A in the specific resin is preferably 1% by mole or more, more preferably 5% by mole or more, still more preferably 10% by mole or more, and particularly preferably 25% by mole or more with respect to all repeating units in the specific resin.

The upper limit of the content of the repeating unit A is not particularly limited, but is preferably 99% by mole or less, more preferably 95% by mole or less, still more preferably 80% by mole or less, and particularly preferably 70% by mole or less with respect to all repeating units in the specific resin.

In a case where the specific resin has two or more kinds of the repeating units A, it is preferable that the total content thereof is within the above-described range.

A structure and a compositional ratio (molar fraction) of each repeating unit contained in the specific resin can be measured by, for example, ¹³C-NMR.

### <Repeating Unit B>

The above-described repeating unit B is a repeating unit having a functional group having a pKa of 10.0 or less, or a salt thereof. The pKa of the above-described functional group is not particularly limited as long as it is 10.0 or less, but is preferably 7.0 or less and more preferably 5.0 or less. The lower limit of the pKa of the above-described functional group is not particularly limited, but is preferably-3.0 or more and more preferably 1.0 or more.

The functional group having a pKa of 10.0 or less is not particularly limited, and examples thereof include an acid group. More specific examples of the functional group having a pKa of 10.0 or less include a carboxy group, a phosphonic acid group, a sulfo group, and a phenolic hydroxy group; and a carboxy group or a sulfo group is preferable, and a carboxy group is more preferable from the viewpoint that solubility of the specific resin in a post-treatment liquid (a rinsing liquid or the like described later) is more excellent.

Examples of the above-described salt include a salt of an acid group. The salt of an acid group refers to a salt in which a hydrogen ion of an acid group is replaced with another cation (for example, a sodium ion).

The number of functional groups having a pKa of 10.0 or less in the repeating unit B is not particularly limited, but is preferably 1 to 4, more preferably 1 to 3, and still more preferably 1 or 2.

Examples of the above-described repeating unit B include a repeating unit represented by Formula (G) and a repeating unit represented by Formula (H).

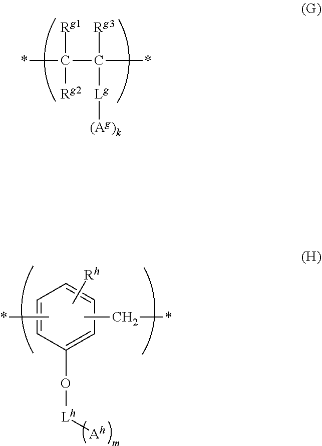

In Formula (G), R^{g1}, R^{g2}, and R^{g3} each independently represent a hydrogen atom, an alkyl group having 1 to 4 carbon atoms, or an acid group.

L^{g} represents a single bond or a (k+1)-valent linking group.

A^{g} represents a functional group having a pKa of 10.0 or less, or a salt thereof.

k represents an integer of 1 to 4.

In a case where a plurality of A^{g}'s are present in Formula (G), the plurality of A^{g}'s may be the same or different from each other.

R^{g1}, R^{g2}, and R^{g3} are preferably a hydrogen atom, a methyl group, an ethyl group, a carboxymethyl group, or a carboxy group, and more preferably a hydrogen atom, a methyl group, or a carboxy group.

Among these, it is preferable that one of R^{g1}, R^{g2}, and R^{g3} represents a hydrogen atom, a methyl group, or a carboxy group, and the remaining two thereof represent a hydrogen atom.

The (k+1)-valent linking group represented by L^{g} is not particularly limited as long as it is a group having a valence corresponding to the number of A^{g}'s; and examples thereof include a di- to pentavalent aliphatic hydrocarbon group which may have a substituent, a di- to pentavalent aromatic ring group which may have a substituent, —O—, —CO—, —SO₂—, —NR^{L}—, —N<, and a group formed by combination of these groups. The definition of R^{L} is as described above.

In a case where k is 1, examples of the divalent linking group include a divalent aliphatic hydrocarbon group, a divalent aromatic ring group, —O—, —CO—, —SO₂—, —NR^{L}—, and a group formed by combination of these groups.

The divalent aliphatic hydrocarbon group may be linear, branched, or cyclic.

As the divalent aliphatic hydrocarbon group, an alkylene group having 1 to 6 carbon atoms (preferably 1 to 3 carbon atoms) is preferable.

In a case where k is 1, L^{g} is preferably a single bond, an alkylene group, a divalent aromatic group, or -alkylene group-NR^{L}-alkylene group, and preferably a single bond, a methylene group, a phenylene group, or -methylene group-NH-methylene group.

A preferred aspect of the functional group having a pKa of 10.0 or less or a salt thereof, which is represented by A^{g}, is as described above.

k is preferably an integer of 1 to 3 and more preferably 1 or 2.

In Formula (H), Rh represents a hydrogen atom or a methyl group.

L^{h} represents a single bond or an (m+1)-valent linking group.

A^{h} represents a functional group having a pKa of 10.0 or less or a salt thereof.

m represents an integer of 1 to 4.

In a case where a plurality of A^{h}'s are present in Formula (H), the plurality of A^{h}'s may be the same or different from each other.

Specific aspects and suitable aspects of the (m+1)-valent linking group represented by L^{h} are the same as the specific aspects and the suitable aspects of the (k+1)-valent linking group represented by L^{g}.

In a case where m is 1, L^{h} is preferably a divalent aliphatic hydrocarbon group, a divalent aromatic group, or -O-divalent aliphatic hydrocarbon group-OCO-divalent aliphatic hydrocarbon group.

The above-described divalent aliphatic hydrocarbon group may be linear, branched, or cyclic.

In addition, a hydrogen atom in the divalent aliphatic hydrocarbon group may be substituted with a substituent such as a hydroxy group.

As the divalent aliphatic hydrocarbon group, among these, an alkylene group or alkenylene group having 1 to 6 carbon atoms is preferable, and an alkylene group or alkenylene group having 1 to 3 carbon atoms is more preferable.

A preferred aspect of the functional group having a pKa of 10.0 or less or a salt thereof, which is represented by A^{h}, is as described above.

m is preferably an integer of 1 to 3 and more preferably 1 or 2.

The specific resin may have only one kind of the above-described repeating unit B or may have two or more kinds thereof.

From the viewpoint that the solubility of the specific resin in the post-treatment liquid is more excellent, a content of the repeating unit B is preferably 1% by mole or more, more preferably 5% by mole or more, still more preferably 20% by mole or more, and particularly preferably 30% by mole or more with respect to all repeating units in the specific resin.

The upper limit thereof is not particularly limited, but is preferably 99% by mole or less, more preferably 95% by mole or less, still more preferably 90% by mole or less, and particularly preferably 75% by mole or less with respect to all repeating units in the specific resin.

A ratio of the repeating unit A and the repeating unit B in the specific resin is not particularly limited, but a ratio (a/b) of a molar number a of the repeating unit A to a molar number b of the repeating unit B is preferably 1/99 or more, more preferably 5/95 or more, still more preferably 10/90 or more, and particularly preferably 25/75 or more.

The lower limit of the ratio (a/b) is not particularly limited, but from the viewpoint that the solubility of the specific resin in the post-treatment liquid is more excellent, it is preferably 99/1 or less, more preferably 95/5 or less, still more preferably 80/20 or less, and particularly preferably 70/30 or less.

In the specific resin, the repeating unit A and the repeating unit B may be randomly bonded (so-called random copolymer), may be alternately bonded (so-called alternating copolymer), or may be bonded in a block manner (so-called block copolymer).

The specific resin may have a repeating unit different from both the repeating unit A and the repeating unit B. A content of the repeating unit different from both the repeating unit A and the repeating unit B in the specific resin is preferably 25% by mole or less, more preferably 0% to 10% by mole, and still more preferably 0% to 5% by mole with respect to all repeating units in the specific resin.

It is preferable that the specific resin does not have a repeating unit different from both the repeating unit A and the repeating unit B.

A weight-average molecular weight Mw of the specific resin is not particularly limited, but is preferably 500 to 1,000,000. Among the above, from the viewpoint that the effect of the present invention is more excellent, the weight-average molecular weight Mw of the specific resin is preferably 1,000 or more, more preferably 2,000 or more, and particularly preferably 5,000 or more. In addition, from the viewpoint that the solubility of the specific resin in the post-treatment liquid is more excellent, the above-described weight-average molecular weight Mw is preferably 500,000 or less, more preferably 100,000 or less, and particularly preferably 50,000 or less.

One kind of the specific resin may be used alone, or two or more kinds thereof may be used in combination.

A content of the specific resin is preferably 1 ppm by mass to 10% by mass, more preferably 10 to 10,000 ppm by mass (1% by mass), still more preferably 50 to 5,000 ppm by mass, and most preferably 50 to 2,000 ppm by mass with respect to the total mass of the present composition.

In addition, in a case where the present composition contains optional components described later, in addition to the specific resin and a solvent, the content of the specific resin is preferably 0.1% to 10.0% by mass and more preferably 0.3% to 3.0% by mass with respect to the total solid content in the present composition.

### [Water]

The present composition contains water.

A content of the water is not particularly limited, but is preferably 1% to 99.9999% by mass, more preferably 50% to 98% by mass, and still more preferably 80% to 97% by mass with respect to the total mass of the present composition.

The water is preferably ultrapure water which is used for manufacturing a semiconductor device.

In particular, the water is preferably water in which inorganic anions, metal ions, and the like are reduced. Among these, the water is more preferably water in which a concentration of ions derived from metal atoms of Fe, Co, Na, K, Ca, Cu, Mg, Mn, Li, Al, Cr, Ni, and Zn is reduced, and still more preferably water in which the concentration is adjusted to be on the order of ppt or less (in one form, a metal content is less than 0.001 ppt by mass) in a case of being used in the preparation of the present composition. The method of carrying out the adjustment is preferably purification using a filtration membrane or an ion-exchange membrane or purification by distillation. Examples of the method of carrying out the adjustment include a method described in paragraphs to of JP2011-110515A and a method described in JP2007-254168A.

The water is preferably water in which the content of each ion is adjusted. In addition, from the viewpoint that the desired effect of the present invention can be remarkably obtained, it is more preferable that the above-described water is used not only for the present composition but also for washing a container. In addition, it is preferable that the above-described water is also used in a step of producing the present composition, measurement of components of the present composition, measurement for evaluation of the present composition, and the like.

### [Optional Component]

The composition may further contain a component other than the above-described components.

Examples of the component which may be contained in the composition include a removing agent, an oxidizing agent, a corrosion inhibitor, a surfactant, an antifoaming agent, and an organic solvent.

### <Removing Agent>

The present composition may contain a removing agent, and it is preferable to contain a removing agent from the viewpoint of further improving residue removability.

The removing agent is not particularly limited as long as it is a compound having a function of removing residues such as etching residues and ashing residues; and examples thereof include a fluorine-containing compound, a hydroxylamine compound, a basic compound, and an acidic compound.

### (Fluorine-Containing Compound)

The fluorine-containing compound is not particularly limited as long as it is a compound containing a fluorine atom, and it may be an inorganic compound containing a fluorine atom or may be an organic compound containing a fluorine atom.

Examples of the fluorine-containing compound include hydrofluoric acid (fluorinated acid), ammonium fluoride, tetramethylammonium fluoride, and tetrabutylammonium fluoride. The fluorine-containing compound has a function of removing residues in the composition. As a result, in a case where the present composition contains a fluorine-containing compound, the residue removability is more excellent.

The fluorine-containing compound is preferably hydrofluoric acid, ammonium fluoride, or tetramethylammonium fluoride, and more preferably hydrofluoric acid or ammonium fluoride.

One kind of the fluorine-containing compound may be used alone, or two or more kinds thereof may be used in combination.

In a case where the present composition contains a fluorine-containing compound, a content of the fluorine-containing compound is preferably 0.01% to 15.0% by mass and more preferably 0.1% to 10.0% by mass with respect to the total mass of the present composition.

### (Hydroxylamine Compound)

The present composition may contain a hydroxylamine compound as the removing agent.

The hydroxylamine compound is at least one compound selected from the group consisting of hydroxylamine (NH₂OH), a hydroxylamine derivative, and salts thereof.

Since the hydroxylamine compound has a function of promoting decomposition and solubilization of residues and removing residues such as etching residues and ashing residues, it is preferable that the present composition contains a hydroxylamine compound as the removing agent.

The hydroxylamine derivative is not particularly limited, and examples thereof include O-methylhydroxylamine, O-ethylhydroxylamine, N-methylhydroxylamine, N,N-dimethylhydroxylamine, N,O-dimethylhydroxylamine, N-ethylhydroxylamine, N,N-diethylhydroxylamine, N,O-diethylhydroxylamine, O,N,N-trimethylhydroxylamine, N,N-dicarboxyethylhydroxylamine, and N,N-disulfoethylhydroxylamine.

Examples of the salts of the hydroxylamine and the hydroxylamine derivative include inorganic acid salts and organic acid salts; and an inorganic acid salt formed by bonding a non-metal atom such as Cl, S, N, and P to a hydrogen atom is preferable, and a salt of any acid of hydrochloric acid, sulfuric acid, or nitric acid is more preferable.

The inorganic acid salts of the hydroxylamine and the hydroxylamine derivative are preferably hydroxylamine nitrate, hydroxylamine sulfate, hydroxylamine hydrochloride, hydroxylamine phosphate, N,N-diethylhydroxylamine sulfate, N,N-diethylhydroxylamine nitrate, or a mixture thereof.

Examples of the organic acid salts of the hydroxylamine and the hydroxylamine derivative include a hydroxylammonium citrate, a hydroxylammonium oxalate, and a hydroxylammonium fluoride.

From the viewpoint of more excellent residue removability, the hydroxylamine compound is preferably hydroxylamine or hydroxylamine sulfate.

One kind of the hydroxylamine compound may be used alone, or two or more kinds thereof may be used in combination.

In a case where the present composition contains a hydroxylamine compound, a content of the hydroxylamine compound is preferably 0.01% to 30% by mass and more preferably 0.5% to 25% by mass, with respect to the total mass of the present composition.

### (Basic Compound)

The present composition may contain a basic compound as the removing agent. The basic compound is intended to be a compound in which a pH of a solution thereof is more than 7 in a case of being dissolved in water. The basic compound also has a function as a pH adjusting agent for adjusting a pH of the present composition.

In the present specification, a compound contained in a corrosion inhibitor described later is not included in the basic compound.

The basic compound may form a salt with an acid group in the repeating unit B of the specific resin.

The basic compound is not particularly limited, and examples thereof include ammonium hydroxide, a water-soluble amine, and a quaternary ammonium compound.

Hereinafter, the ammonium hydroxide, the water-soluble amine, and the quaternary ammonium compound will be described in detail.

The present composition may contain ammonium hydroxide (NH₄OH) as the basic compound.

In a case where the present composition contains ammonium hydroxide, a content of the ammonium is preferably 0.01% to 15.0% by mass and more preferably 0.05% to 10.0% by mass with respect to the total mass of the present composition.

The present composition may contain a water-soluble amine as the basic compound. In the present specification, the water-soluble amine is intended to be a compound having at least one group selected from the group consisting of a primary amino group, a secondary amino group, and a tertiary amino group in the molecule, and capable of being dissolved in 50 g or more in 1 L of water.

Examples of the water-soluble amine include a primary amine having a primary amino group in the molecule, a secondary amine having a secondary amino group in the molecule, a tertiary amine having a tertiary amino group in the molecule, and salts thereof.

Examples of the salts of the above-described amines include a salt of an inorganic acid, in which at least one non-metal selected from the group consisting of Cl, S, N, and P is bonded to hydrogen; and a hydrochloride, a sulfate, or a nitrate is preferable.

The water-soluble amine is preferably a low-molecular-weight compound. In the present specification, the “low-molecular-weight compound” means a compound having substantially no molecular weight distribution. The molecular weight of the low-molecular-weight compound is preferably 1,000 or less. Any of following specific examples of water-soluble amines is the low-molecular-weight compound having a molecular weight of 1,000 or less.

The water-soluble amine may be an alicyclic amine compound having a ring structure in the molecule or an alkanol amine having at least one hydroxyalkyl group in the molecule.

One kind of the water-soluble amine may be used alone, or two or more kinds thereof may be used in combination.

In a case where the present composition contains a water-soluble amine, a content of the water-soluble amine is preferably 0.01% to 10% by mass and more preferably 0.1% to 5.0% by mass with respect to the total mass of the present composition.

The present composition may contain, as the removing agent, a quaternary ammonium compound having one quaternary ammonium cation group in the molecule.

The quaternary ammonium compound is not particularly limited as long as it is a compound having at least one quaternary ammonium cationic group in which a nitrogen atom is substituted with four hydrocarbon groups (preferably, alkyl groups).

Examples of the quaternary ammonium compound include a quaternary ammonium hydroxide, a quaternary ammonium fluoride, a quaternary ammonium bromide, a quaternary ammonium iodide, a quaternary ammonium acetate, and a quaternary ammonium carbonate. The quaternary ammonium compound is preferably a quaternary ammonium hydroxide and more preferably a compound represented by Formula (a1).

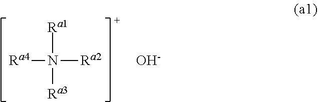

In Formula (a1), R^{a1} to R^{a4} each independently represent an alkyl group having 1 to 16 carbon atoms, an aryl group having 6 to 16 carbon atoms, an aralkyl group having 7 to 16 carbon atoms, or a hydroxyalkyl group having 1 to 16 carbon atoms. At least two of R^{a1} to R^{a4} may be bonded to each other to form a ring structure.

From the viewpoint of ease of availability, the above-described compound represented by Formula (a1) is preferably at least one selected from the group consisting of tetramethylammonium hydroxide (TMAH), tetraethylammonium hydroxide (TEAH), tetrapropylammonium hydroxide, tetrabutylammonium hydroxide (TBAH), methyltripropylammonium hydroxide, methyltributylammonium hydroxide, ethyltrimethylammonium hydroxide, dimethyldiethylammonium hydroxide, benzyltrimethylammonium hydroxide (BzTMAH), hexadecyltrimethylammonium hydroxide, (2-hydroxyethyl)trimethylammonium hydroxide, and spiro-(1,1′)-bipyrrolidinium hydroxide; more preferably TMAH, TEAH, TBAH, or BzTMAH; and still more preferably TMAH, TEAH, or TBAH.

One kind of the quaternary ammonium compound may be used alone, or two or more kinds thereof may be used in combination.

In a case where the present composition contains a quaternary ammonium compound, a content of the quaternary ammonium compound is preferably 0.01% to 15% by mass and more preferably 0.1% to 10% by mass with respect to the total mass of the present composition.

One kind of the basic compound may be used alone, or two or more kinds thereof may be used in combination.

In a case where the present composition contains a basic compound, a content of the basic compound is preferably 0.01% to 20% by mass and more preferably 0.01% to 10% by mass with respect to the total mass of the present composition.

### (Acidic Compound)

The present composition may contain an acidic compound as the removing agent. The acidic compound is intended to be a compound in which a pH of a solution thereof is less than 7 in a case of being dissolved in water. The acidic compound also has a function as a pH adjusting agent for adjusting the pH of the present composition.

In the present specification, a compound included in any of an oxidizing agent described later or an anionic surfactant described later is not included in the acidic compound.

The acidic compound may be an inorganic acid or an organic acid.

As described above, the inorganic acid or the organic acid may form a salt with the specific group in the repeating unit A of the specific resin.

Examples of the inorganic acid include nitric acid, sulfuric acid, hydrochloric acid, and phosphoric acid, and sulfuric acid is preferable.

One kind of the inorganic acid may be used alone, or two or more kinds thereof may be used in combination.

In a case where the present composition contains an inorganic acid, a content of the inorganic compound is preferably 0.01% to 20% by mass and more preferably 0.01% to 10% by mass with respect to the total mass of the present composition.

The organic acid is an organic compound which has an acidic functional group and is acidic (has a pH of less than 7.0) in an aqueous solution. Examples of the acidic functional group include a carboxy group, a phosphonic acid group, a sulfo group, and a phenolic hydroxy group.

The organic acid is not particularly limited, and examples thereof include a carboxylic acid having a carboxy group in the molecule (organic carboxylic acid), a phosphonic acid having a phosphonic acid group in the molecule (organic phosphonic acid), and a sulfonic acid having a sulfo group in the molecule (organic sulfonic acid); and an organic carboxylic acid group is preferable.

The number of acidic functional groups in the organic acid is not particularly limited, but is preferably 1 to 4 and more preferably 1 to 3.

In addition, the organic acid is preferably a compound having a function of chelating with a metal contained in the residues, and preferably a compound having two or more functional groups (coordinating groups) which forms a coordinate bond with a metal ion in the molecule. Examples of the coordinating group include the above-described acidic functional group and specific group.

Examples of the carboxylic acid include a polyamino polycarboxylic acid, an amino acid, a polycarboxylic acid, and a monocarboxylic acid.

The phosphonic acid may be a monophosphonic acid having one phosphonic acid group in the molecule, or may be a polyphosphonic acid having two or more phosphonic acid groups in the molecule.

The number of phosphonic acid groups in the phosphonic acid is preferably 2 to 5, more preferably 2 to 4, and still more preferably 2 or 3.

The sulfonic acid may be a monosulfonic acid having only one sulfo group in the molecule, or may be a polysulfonic acid having two or more sulfo groups in the molecule.

The number of sulfo groups in the sulfonic acid is preferably 1 or 2 and more preferably 1.

Examples of the sulfonic acid include methanesulfonic acid (MSA), ethanesulfonic acid, isethionic acid (2-hydroxyethanesulfonic acid), benzenesulfonic acid, and p-toluenesulfonic acid (tosylic acid); and methanesulfonic acid or isethionic acid is preferable.

The organic acid preferably has a low molecular weight. Specifically, the molecular weight of the organic acid is preferably 600 or less and more preferably 450 or less. The lower limit thereof is not particularly limited, but is preferably 85 or more.

In addition, the number of carbon atoms in the organic acid is preferably 15 or less, more preferably 12 or less, and still more preferably 8 or less. The lower limit thereof is not particularly limited, but is preferably 2 or more.

The organic acid is preferably the above-described carboxylic acid; more preferably the above-described polyamino polycarboxylic acid, the above-described amino acid, or the above-described polycarboxylic acid; and still more preferably the above-described amino acid or the above-described polycarboxylic acid.

One kind of the organic acid may be used alone, or two or more kinds thereof may be used in combination.

In a case where the present composition contains an organic acid, a content of the organic acid is preferably 0.001% to 20% by mass and more preferably 0.005% to 10% by mass with respect to the total mass of the present composition.

The present composition may contain a removing agent other than those described above. Examples of other removing agents include a compound having at least two nitrogen-containing groups and having no carboxy group. Specific examples of such a compound include at least one biguanide compound selected from the group consisting of a compound having a biguanide group and a salt thereof.

In addition, as the removing agent, a chelating agent described in JP2017-504190A can also be used, and the content described in this document is incorporated in the present specification.

The removing agent is preferably at least one selected from the group consisting of a fluorine-containing compound, a hydroxylamine compound, a basic compound, and an acidic compound; and more preferably at least one selected from the group consisting of hydrofluoric acid, ammonium fluoride, a hydroxylamine compound, ammonium hydroxide, water-soluble amine, a quaternary ammonium compound, sulfuric acid, and organic carboxylic acid.

In addition, from the viewpoint of further improving removability of dry etching residues in a case of being used as a washing solution, the present composition preferably contains, as the removing agent, at least one selected from the group consisting of hydrofluoric acid, a hydroxylamine compound, ammonium hydroxide, water-soluble amine, and a quaternary ammonium compound, and more preferably contains hydroxylamine, ammonium hydroxide, TMAH, or hydrofluoric acid.

One kind of the removing agent may be used alone, or two or more kinds thereof may be used in combination.

In a case where the composition contains a removing agent, a content of the removing agent is preferably 0.001% to 20% by mass and more preferably 0.005% to 10% by mass with respect to the total mass of the present composition.

In addition, the content of the removing agent is preferably 0.1% to 98.0% by mass and more preferably 0.3% to 85.0% by mass with respect to the total solid content in the present composition.

### <Oxidizing Agent>

The present composition may contain an oxidizing agent. In a case where the present composition is an etchant, the present composition preferably contains an oxidizing agent.

Examples of the oxidizing agent include a peroxide such as hydrogen peroxide and peracetic acid, nitric acid, iodic acid, periodic acid, hypochlorous acid, chlorous acid, chloric acid, perchloric acid, persulfuric acid, bichromic acid, permanganic acid, ozone water, a silver (II) salt, and an iron (III) salt such as iron nitrate. The above-described oxidizing agent may form a salt with a counter ion.

The oxidizing agent contained in the present composition is preferably hydrogen peroxide, nitric acid, peracetic acid, periodic acid, perchloric acid, chloric acid, hypochlorous acid, a cerium ammonium nitrate salt, iron nitrate, or ammonium persulfate; and more preferably hydrogen peroxide, nitric acid, peracetic acid, periodic acid, or perchloric acid.

One kind of the oxidizing agent may be used alone, or two or more kinds thereof may be used in combination.

In a case where the present composition contains an oxidizing agent, a content of the oxidizing agent is preferably 0.1% to 20% by mass and more preferably 0.5% to 15% by mass with respect to the total mass of the present composition.

In addition, the content of the oxidizing agent is preferably 10% to 80% by mass and more preferably 30% to 60% by mass with respect to the total solid content in the present composition.

### <Corrosion Inhibitor>

The present composition may contain a corrosion inhibitor, and it is preferable to contain a corrosion inhibitor.

The corrosion inhibitor is not particularly limited as long as it is a compound which has a function of preventing corrosion of a metal-containing layer due to over-etching or the like, by being coordinated to form a film on the surface of the metal-containing layer; and examples thereof include a heteroaromatic compound, a thiol compound, and a catechol compound.

The heteroaromatic compound is not particularly limited as long as it is a compound having a heteroaromatic ring structure in the molecule, but it is preferably a nitrogen-containing heteroaromatic compound in which at least one of heteroatoms constituting the heteroaromatic ring is a nitrogen atom.

Examples of the nitrogen-containing heteroaromatic compound include an azole compound, a pyridine compound, a pyrazine compound, and a pyrimidine compound; and an azole compound is preferable.

The azole compound is a compound which has a hetero 5-membered ring containing one or more nitrogen atoms and has aromaticity. The number of nitrogen atoms in the hetero 5-membered ring of the azole compound is preferably 1 to 4 and more preferably 1 to 3.

The azole compound may have a substituent on the hetero 5-membered ring. Examples of the above-described substituent include a hydroxy group, a carboxy group, a mercapto group, an amino group, an alkyl group having 1 to 4 carbon atoms, which may have an amino group, and a 2-imidazolyl group.

Examples of the azole compound include an imidazole compound in which one of atoms constituting the azole ring is a nitrogen atom, a pyrazole compound in which two of atoms constituting an azole ring are nitrogen atoms, and a thiazole compound in which one of atoms constituting an azole ring is a nitrogen atom and the other is a sulfur atom, a triazole compound in which three of atoms constituting an azole ring are nitrogen atoms, and a tetrazole compound in which four of atoms constituting an azole ring are nitrogen atoms.

Examples of the imidazole compound include imidazole, 1-methylimidazole, 2-methylimidazole, 5-methylimidazole, 1,2-dimethylimidazole, 2-mercaptoimidazole, 4,5-dimethyl-2-mercaptoimidazole, 4-hydroxyimidazole, 2,2′-biimidazole, 4-imidazole carboxylic acid, histamine, and benzimidazole.

Examples of the pyrazole compound include 2,4-dimethylthiazole, benzothiazole, and 2-mercaptobenzothiazole.

Examples of the thiazole compound include 2,4-dimethylthiazole, benzothiazole, and 2-mercaptobenzothiazole.

Examples of the triazole compound include 1,2,4-triazole, 3-methyl-1,2,4-triazole, 3-amino-1,2,4-triazole, 1,2,3-triazole, 1-methyl-1,2,3-triazole, benzotriazole, 1-hydroxybenzotriazole, 1-dihydroxypropylbenzotriazole, 2,3-dicarboxypropylbenzotriazole, 4-hydroxybenzotriazole, 4-carboxybenzotriazole, 5-methyl-1H-benzotriazole, and 2,2′-{[(5-methyl-1H-benzotriazole-1-yl)methyl]imino}diethanol. Among these, benzotriazole, 5-methyl-1H-benzotriazole, or tolyl triazole is preferable, and 5-methyl-1H-benzotriazole is more preferable.

Examples of the tetrazole compound include 1H-tetrazole (1,2,3,4-tetrazole), 5-methyl-1,2,3,4-tetrazole, 5-amino-1,2,3,4-tetrazole, 1,5-pentamethylenetetrazole, 5-mercapto-1-phenyltetrazole, and 1-(2-dimethylaminoethyl)-5-mercaptotetrazole. Among these, 5-mercapto-1-phenyltetrazole is preferable.

The pyridine compound is a compound which has a hetero 6-membered ring (pyridine ring) containing one nitrogen atom and having aromaticity; the pyrazine compound is a compound which has a hetero 6-membered ring (pyrazine ring) having aromaticity and containing two nitrogen atoms located at the para position; and the pyrimidine compound is a compound which has a hetero 6-membered ring (pyrimidine ring) having aromaticity and containing two nitrogen atoms located at the meta position.

The thiol compound means a compound having at least one thiol group and a hydrocarbon group.

The number of thiol groups in the thiol compound is not particularly limited, but is preferably 1 or 2 and more preferably 1.

Examples of the hydrocarbon group in the thiol compound include an alkyl group (preferably having 4 to 20 carbon atoms), an alkenyl group (preferably having 4 to 12 carbon atoms), an alkynyl group (preferably having 4 to 12 carbon atoms), an aryl group (preferably having 6 to 14 carbon atoms), and an aralkyl group (preferably having 7 to 16 carbon atoms).

The hydrocarbon group may have a substituent. Examples of the substituent include a hydroxyl group, a carboxy group, and an amino group which may have an alkyl group.

The catechol compound means at least one selected from the group consisting of pyrocatechol (benzene-1,2-diol) and a catechol derivative.

The catechol derivative means a compound in which pyrocatechol is substituted with at least one substituent. Examples of the substituent in the catechol derivative include a hydroxy group, a carboxy group, a carboxylic acid ester group, a sulfo group, a sulfonic acid ester group, an alkyl group (preferably having 1 to 6 carbon atoms), and an aryl group (preferably, a phenyl group). The carboxy group and the sulfo group, which are included in the catechol derivative as a substituent, may be a salt with a cation. In addition, the alkyl group and the aryl group, which are included in the catechol derivative as a substituent, may further have a substituent.

The corrosion inhibitor is preferably a heteroaromatic compound or a thiol compound, and more preferably a triazole compound, a tetrazole compound, or a thiol compound.

One kind of the corrosion inhibitor may be used alone, or two or more kinds thereof may be used in combination.

In a case where the present composition contains a corrosion inhibitor, a content of the corrosion inhibitor is preferably 0.001% to 10% by mass, more preferably 0.002% to 5% by mass, and still more preferably 0.03% to 1% by mass with respect to the total mass of the present composition.

In addition, the content of the corrosion inhibitor is preferably 0.1% to 10.0% by mass and more preferably 0.5% to 10.0% by mass with respect to the total solid content in the present composition.

As the corrosion inhibitor, a corrosion inhibitor of a high-purity grade is preferably used, which is more preferably used by being further purified.

A method of purifying the corrosion inhibitor is not particularly limited, but for example, a well-known method such as filtration, ion exchange, distillation, adsorption purification, recrystallization, reprecipitation, sublimation, and purification using a column is used, and this method can be also applied in combination.

### <Surfactant>

The present composition may contain a surfactant.

From the viewpoint that the dissolution of the metal film can be further suppressed, it is preferable that the composition contains a surfactant.

The surfactant is not particularly limited as long as it is a compound having a hydrophilic group and a hydrophobic group (lipophilic group) in the molecule; and examples thereof include an anionic surfactant, a cationic surfactant, a nonionic surfactant, and an amphoteric surfactant.

In a large number of cases, the surfactant has a hydrophobic group selected from an aliphatic hydrocarbon group, an aromatic hydrocarbon group, or a combination thereof. The hydrophobic group in the surfactant is not particularly limited, but in a case where the hydrophobic group includes an aromatic hydrocarbon group, the number of carbon atoms in the hydrophobic group is preferably 6 or more, and more preferably 10 or more. In a case where the hydrophobic group does not include an aromatic hydrocarbon group and is composed only of an aliphatic hydrocarbon group, the number of carbon atoms in the hydrophobic group is preferably 10 or more, and more preferably 12 or more. The upper limit of the number of carbon atoms in the hydrophobic group is not particularly limited, but is preferably 20 or less.

### (Anionic Surfactant)

Examples of the anionic surfactant contained in the present composition include a phosphoric acid ester-based surfactant, a phosphonic acid-based surfactant, a sulfonic acid-based surfactant, a carboxylic acid-based surfactant, and a sulfuric acid ester-based surfactant.

The anionic surfactant is preferably a phosphoric acid ester-based surfactant, a sulfonic acid-based surfactant, a carboxylic acid-based surfactant, or a sulfuric acid ester-based surfactant.

### (Cationic Surfactant)

Examples of the cationic surfactant include primary to tertiary alkylamine salts (for example, monostearylammonium chloride, distearylammonium chloride, and tristearylammonium chloride), quaternary ammonium salts (for example, dodecyltrimethylammonium chloride), and modified aliphatic polyamines (for example, polyethylene polyamine).

### (Nonionic Surfactant)

Examples of the nonionic surfactant include a polyoxyalkylene alkyl ether, a polyoxyalkylene alkenyl ether, a polyoxyethylene alkyl phenyl ether, a polyoxyalkylene glycol, a polyoxyalkylene monoalkylate, a polyoxyalkylene dialkylate, a bispolyoxyalkylene alkylamide, a sorbitan fatty acid ester, a polyoxyethylene sorbitan fatty acid ester, a polyoxyethylene alkylamine, a glycerin fatty acid ester, an oxyethyleneoxypropylene block copolymer, an acetylene glycol-based surfactant, and an acetylene-based polyoxyethylene oxide.

### (Amphoteric Surfactant)

Examples of the amphoteric surfactant include carboxybetaine (for example, an alkyl-N,N-dimethylaminoacetic acid betaine and an alkyl-N,N-dihydroxyethylaminoacetic acid betaine), sulfobetaine (for example, an alkyl-N,N-dimethylsulfoethyleneammonium betaine), and imidazolinium betaine (for example, a 2-alkyl-N-carboxymethyl-N-hydroxyethyl imidazolinium betaine).

As the surfactant, compounds described in paragraphs to of JP2015-158662A, paragraphs and of JP2012-151273A, and paragraphs to of JP2009-147389A can also be used, the contents of which are incorporated herein by reference.

One kind of the surfactant may be used alone, or two or more kinds thereof may be used in combination.

In a case where the present composition contains a surfactant, from the viewpoint that the effect of the present invention is more excellent, a content of the surfactant is preferably 0.001% to 3% by mass and more preferably 0.005% to 2% by mass with respect to the total mass of the present composition.

In addition, the content of the surfactant is preferably 1.0% to 40.0% by mass and more preferably 5.0% to 30.0% by mass with respect to the total solid content in the present composition.

### <Antifoaming Agent>

The composition may contain an antifoaming agent. The surfactant may cause foaming depending on the usage. As a result, it is preferable that the composition containing the surfactant contains an antifoaming agent which suppresses the occurrence of foaming, shortens the life of the generated foam, and suppresses foam residues.

The antifoaming agent is not particularly limited as long as the effect of the present invention is not impaired; and examples thereof include a silicone-based antifoaming agent, an acetylenediol-based antifoaming agent, a fatty acid ester-based antifoaming agent, and a long-chain aliphatic alcohol-based antifoaming agent. Among these, a silicone-based antifoaming agent is preferable from the viewpoint that the effect of suppressing foam residues is more excellent.

The antifoaming agent does not include the compound included in the surfactant described above.

One kind of the antifoaming agent may be used alone, or two or more kinds thereof may be used in combination.

In a case where the present composition contains an antifoaming agent, from the viewpoint that the suppression of foam residues is more excellent, a content of the antifoaming agent is preferably 0.0001% to 3% by mass and more preferably 0.001% to 2% by mass with respect to the total mass of the present composition.

In addition, the content of the antifoaming agent is preferably 1.0% to 40.0% by mass and more preferably 5.0% to 30.0% by mass with respect to the total solid content in the present composition.

### <Organic Solvent>

The present composition may contain an organic solvent, and it is preferable to contain an organic solvent.

The organic solvent is preferably a water-soluble organic solvent. The fact that the organic solvent is water-soluble is intended to be that the organic solvent can be mixed with (dissolved in) water at 25° C. with arbitrary ratio.

Examples of the organic solvent include an alcohol-based solvent, a ketone-based solvent, an ester-based solvent, an ether-based solvent (for example, a glycol diether), a sulfone-based solvent, a sulfoxide-based solvent, a nitrile-based solvent, and an amide-based solvent.

These solvents may be water-soluble.

Among these, the present composition preferably contains one or more kinds of organic solvents selected from the group consisting of an alcohol-based solvent, a ketone-based solvent, an ester-based solvent, and an ether-based solvent.

Examples of the alcohol-based solvent include an alkanediol (including, for example, an alkylene glycol), an alkoxyalcohol (including, for example, a glycol monoether), a saturated aliphatic monohydric alcohol, an unsaturated non-aromatic monohydric alcohol, and a low-molecular-weight alcohol containing a ring structure.

Examples of the alkanediol include glycol, 2-methyl-1,3-propanediol, 1,3-propanediol, 2,2-dimethyl-1,3-propanediol, 1,4-butanediol, 1,3-butanediol, 1,2-butanediol, 2,3-butanediol, pinacol, and an alkylene glycol.

Examples of the alkylene glycol include ethylene glycol, propylene glycol, hexylene glycol, diethylene glycol, dipropylene glycol, triethylene glycol, and tetraethylene glycol; and propylene glycol or hexylene glycol is preferable.

Examples of the alkoxyalcohol include 3-methoxy-3-methyl-1-butanol, 3-methoxy-1-butanol, 1-methoxy-2-butanol, and a glycol monoether; and a glycol monoether is preferable.

Examples of the glycol monoether include ethylene glycol mono C₁ to C₄ alkyl ethers such as ethylene glycol monomethyl ether, ethylene glycol monoethyl ether, ethylene glycol mono-n-propyl ether, ethylene glycol monoisopropyl ether, and ethylene glycol monobutyl ether; diethylene glycol mono C₁ to C₄ alkyl ethers such as diethylene glycol monomethyl ether, diethylene glycol monoethyl ether, and diethylene glycol monobutyl ether; triethylene glycol mono C₁ to C₄ alkyl ethers such as triethylene glycol monomethyl ether, triethylene glycol monoethyl ether, and triethylene glycol monobutyl ether; and 1-methoxy-2-propanol, 2-methoxy-1-propanol, 1-ethoxy-2-propanol, 2-ethoxy-1-propanol, propylene glycol mono-n-propyl ether, dipropylene glycol monomethyl ether, dipropylene glycol monoethyl ether, dipropylene glycol mono-n-propyl ether, tripropylene glycol monoethyl ether, tripropylene glycol monomethyl ether, ethylene glycol monobenzyl ether, and diethylene glycol monobenzyl ether.

Among these, an ethylene glycol mono C₁ to C₄ alkyl ether is preferable, and ethylene glycol monomethyl ether, ethylene glycol monoethyl ether, or ethylene glycol monobutyl ether is more preferable.

Examples of the saturated aliphatic monohydric alcohol include methanol, ethanol, n-propyl alcohol, isopropyl alcohol, 1-butanol, 2-butanol, isobutyl alcohol, tert-butyl alcohol, 2-pentanol, t-pentyl alcohol, and hexanol.

Examples of the unsaturated non-aromatic monohydric alcohol include allyl alcohol, propargyl alcohol, 2-butenyl alcohol, 3-butenyl alcohol, and 4-penten-2-ol.

Examples of the low-molecular-weight alcohol containing a ring structure include tetrahydrofurfuryl alcohol, furfuryl alcohol, and 1,3-cyclopentanediol.

One kind of the organic solvent may be used alone, or two or more kinds thereof may be used in combination.

In a case where the present composition contains an organic solvent, a content of the organic solvent is preferably 0.1% to 30% by mass and more preferably 1% to 15% by mass with respect to the total mass of the present composition.

### <Metal Component>

The present composition may contain a metal component.

Examples of the metal component include metal particles and metal ions. For example, as a content of the metal component, it indicates the total content of metal particles and metal ions.

The composition may contain metal particles or metal ions, or may contain both metal particles and metal ions.

Examples of the metal atom contained in the metal component include an metal atom selected from the group consisting of A^{g}, Al, As, Au, Ba, Ca, Cd, Co, Cr, Cu, Fe, Ga, Ge, K, Li, Mg, Mn, Mo, Na, Ni, Pb, Sn, Sr, Ti, and Zn.

The metal component may contain one metal atom or two or more metal atoms.

The metal particles may be a simple body or an alloy, or may be present in a form in which the metal is associated with an organic substance.

The metal component may be a metal component unavoidably contained in each component (raw material) contained in the composition, or may be a metal component unavoidably contained during the production, storage, and/or transfer of the composition, and the metal component may be added intentionally.

In a case where the present composition contains a metal component, a content of the metal component is usually 0.01 ppt by mass to 10 ppm by mass, preferably 0.1 ppt by mass to 1 ppm by mass and more preferably 0.1 ppt by mass to 100 ppb by mass with respect to the total mass of the present composition.

The kind and content of the metal component in the present composition can be measured by a single nano particle inductively coupled plasma mass spectrometry (SP—ICP-MS method). The SP—ICP-MS method is different from a general inductively coupled plasma mass spectrometry (ICP-MS method) only in data analysis, but uses the same apparatus as in the general ICP-MS method. The data analysis of the SP—ICP-MS method can be carried out using commercially available software.

In the ICP-MS method, the content of the metal component to be measured is measured regardless of the existence form thereof. As a result, the total mass of metal particles and metal ions to be measured is quantified as the content of the metal component. On the other hand, in the SP—ICP-MS method, the content of the metal particles can be measured. As a result, the content of the metal ions in the sample can be calculated by subtracting the content of the metal particles from the content of the metal component in the sample.

Regarding a measuring method by the SP—ICP-MS method, it is possible to carry out measurement according to a method described in Examples, for example, by Agilent 8800 triple quadrupole inductively coupled plasma mass spectrometry (ICP-MS, for semiconductor analysis, option #200) manufactured by Agilent Technologies, Inc. As an apparatus other than the above-described apparatus, NexION350S manufactured by Perkin Elmer, Inc. or Agilent 8900 manufactured by Agilent Technologies, Inc. can also be used.

A method of adjusting the content of each metal component in the present composition is not particularly limited. For example, the content of the metal component in the present composition can be reduced by carrying out a well-known treatment of removing a metal from the present composition and/or from a raw material containing each component which is used in the preparation of the present composition. In addition, the content of the metal component in the present composition can be increased by adding a compound containing metal ions to the composition.

The present composition may contain an additive other than the above-described components. Examples of the additive include an antibacterial agent, a rust inhibitor, and a preservative.

The content of each of the components (excluding the metal component) in the present composition can be measured by a well-known method such as gas chromatography-mass spectrometry (GC-MS), liquid chromatography-mass spectrometry (LC-MS), and ion-exchange chromatography (IC).

### [Physical Properties of Composition]

<pH>

A pH of the present composition is not particularly limited, and is preferably 2.0 to 12.0. In a case where the present composition is used as a washing solution, the pH of the present composition is more preferably 3.0 to 10.0 and still more preferably 8.0 to 10.0. This is because the effect of the present invention is more excellent in the composition which has a pH in the above-described range.

The pH of the present composition is a value obtained by carrying out measurement at 25° C. in accordance with JIS Z8802-1984 using a pH meter (for example, manufactured by HORIBA, Ltd., model “F-74”).

### <Coarse Particles>

It is preferable that the present composition is substantially free of coarse particles.

The coarse particles refer to particles having a diameter of 0.2 μm or more in a case where a shape of the particles is regarded as a sphere. In addition, the fact of being substantially free of coarse particles refers to that ten or less particles of 0.2 μm or more are present in 1 mL of the composition, in a case where the composition is subjected to measurement using a commercially available measuring device in the light scattering type in-liquid particle measuring method.

The coarse particles contained in the present composition are particles such as dirt, dust, organic solids, and inorganic solids, which are contained as impurities in raw materials, and particles such as dirt, dust, organic solids, and inorganic solids, which are brought in as contaminants during the preparation of the composition, and the coarse particles correspond to particles which are finally present as particles without being dissolved in the present composition.

The amount of the coarse particles present in the present composition can be measured in a liquid phase using a commercially available measuring device in the light scattering type in-liquid particle measuring method, using laser as a light source.

Examples of a method of removing the coarse particles include a treatment such as filtering.

### [Kit and Concentrated Solution]

The raw materials of the present composition may be divided into a plurality of parts to be used as a kit for preparing the present composition. Although not particularly limited, specific examples of the method using the present composition as a kit include an aspect in which a composition containing water and a removing agent as a first liquid is prepared, and a composition containing the specific resin as a second liquid is prepared.

A content of each component contained in the first liquid and the second liquid provided in the kit is not particularly limited, but the content of each component in the present composition prepared by mixing the first liquid and the second liquid is preferably an amount corresponding to the preferred amount described above.

A pH of each of the first liquid and the second liquid provided in the kit is not particularly limited, and it is sufficient that the pH is adjusted such that the pH of the present composition prepared by mixing the first liquid and the second liquid is a desired value.

In addition, the present composition may be prepared as a concentrated solution. In this case, a diluent liquid obtained by diluting the composition with a liquid for dilution is used. That is, the kit may be a kit containing the above-described composition as a form of a concentrated solution and the above-described liquid for dilution.

The liquid for dilution is preferably a liquid selected from the group consisting of water, isopropanol, a mixed liquid of water and isopropanol, and a solvent containing ammonium hydroxide; more preferably water, isopropanol, or a mixed liquid of water and isopropanol; and still more preferably water.

A dilution ratio of the present composition is not particularly limited, but is preferably 1 to 2,000 times and more preferably 1 to 100 times.

It is also possible to suitably use a composition (hereinafter, also referred to as “diluent liquid”) containing each component which can be contained in the present composition with an amount obtained by dividing a suitable content of each component (excluding the water) by a dilution ratio (for example, 100) in the above-described range.

The suitable content of each component (excluding the water) with respect to the total mass of the diluent liquid is an amount obtained, for example, by dividing the amount described as the suitable content of each component with respect to the total mass of the composition before dilution by a dilution ratio in the above-described range (for example, 100).

A specific method of a diluting step of diluting the present composition may be carried out according to a composition preparation step described later. For a stirring device and a stirring method, which are used in the diluting step, a well-known stirring device described in the composition preparation step described later may be used.

### [Use Application]

Next, use application of the composition according to the above-described embodiment will be described.

The present composition is a composition for a semiconductor device. In the present specification, the “for a semiconductor device” means that it is used in the manufacture of a semiconductor device. The present composition can be used in any step for manufacturing a semiconductor device, and for example, it can be used in a step of treating a semiconductor substrate, which is included in a manufacturing method of a semiconductor device.

More specifically, the present composition can be used for treating an insulating film, a resist, an antireflection film, etching residues (particularly, dry etching residues), ashing residues, and residues derived from a resist film such as a photoresist and a metal hard mask, and the like on a substrate. In the present specification, the etching residues, the ashing residues, the residues derived from a resist film, and the like are collectively referred to as residues. In addition, the present composition may be used in an etching treatment for removing a metal-containing substance on a substrate, or may be used for treating a substrate after chemical mechanical polishing.

For example, the present composition can be used as a pre-wet liquid to be applied on a substrate to improve coatability of an actinic ray-sensitive or radiation-sensitive composition before the step of forming a resist film using the composition, a washing solution that is used for removing residues which have adhered on a metal layer, a solution (for example, a removal liquid, a stripping liquid, or the like) that is used for removing various resist films for pattern formation, a solution (for example, a removal liquid, a stripper, or the like) that is used for removing a permanent film (for example, a color filter, a transparent insulating film, a lens made of a resin) or the like from a semiconductor substrate, or the like.

Since the semiconductor substrate after the removal of the permanent film may be used again for the semiconductor device, the removal of the permanent film is included in the manufacturing process of the semiconductor device.

In addition, the present composition can also be used as a washing solution that is used for removing residues such as metal impurities and fine particles from a substrate after chemical mechanical polishing.

In addition, the present composition can also be used as an etchant for a metal-containing substance (including a metal oxide and a composite oxide consisting of a plurality of metal oxides) on a substrate.

Among the above-described use applications, in particular, the present composition can be suitably used as a washing solution for removing residues, a solution for removing a resist film used in a pattern formation, a washing solution for removing residues from a substrate after chemical mechanical polishing, or an etchant.

Among the above-described use applications, the present composition may be used in only one use application or two or more of use applications.

In the above-described use applications, a diluent liquid obtained by diluting the present composition can also be used. Among these, the present composition can be suitably used as a washing solution for removing residues on a substrate (more preferably, a substrate on which chemical mechanical polishing has been carried out).

The present composition can be suitably used for treating a substrate of a semiconductor device, which includes a metal layer containing tungsten (W), and a substrate of a semiconductor device, which includes a metal layer containing Mo. The present composition can also be used for treating a substrate of a semiconductor device, which includes a metal layer containing Co, and a substrate of a semiconductor device, which includes a metal layer containing Cu.

Furthermore, since the present composition has an excellent corrosion prevention property with respect to an insulating film, the present composition can also be used, for example, for treating a substrate of a semiconductor device, which includes a layer containing at least one selected from the group consisting of SiOx, SiN, and SiOC (x represents a number of 1 to 3).

### [Composition, Concentrated Solution, and Production Method of Kit]

### <Composition Preparation Step>

A production method of the present composition is not particularly limited, and the present composition can be produced by a well-known production method. Examples of the production method of the present composition include a method including at least a composition preparation step of mixing the above-described respective components to prepare the composition.

In the composition preparation step, the order in which the respective components are mixed is not particularly limited. It is preferable that each of the liquids provided in the kit and the concentrated solution is also produced according to the same method as described above.

A production method of the kit is not particularly limited. For example, after preparing the first liquid and the second liquid described above, the first liquid and the second liquid may be respectively accommodated in containers different from each other to produce a kit for preparing the composition.

### <Filtration Step>

It is preferable that the above-described production method includes a filtration step of filtering the liquid in order to remove foreign substances, coarse particles, and the like from the liquid.

The filtration method is not particularly limited, and a well-known filtration method can be used. Among these, filtering using a filter is preferable.

The filter used for the filtering can be used without particular limitation as long as it is a filter which is conventionally used in the use application of the filtering. Examples of a material constituting the filter include a fluororesin such as polytetrafluoroethylene (PTFE), a polyamide-based resin such as nylon, and a polyolefin-based resin (having a high density and an ultrahigh molecular weight) such as polyethylene and polypropylene (PP). Among these, a polyamide-based resin, PTFE, or polypropylene (including high-density polypropylene) is preferable.

In a case of using a filter formed from these materials, it is possible to more effectively remove, from the composition, foreign substances having high polarity, which are likely to cause defects.

A lower limit value of a critical surface tension of the filter is preferably 70 mN/m or more, and an upper limit value thereof is preferably 95 mN/m or less. In particular, the critical surface tension of the filter is preferably 75 to 85 mN/m.

The value of the critical surface tension is a nominal value of a manufacturer. In a case of using a filter having a critical surface tension within the above-described range, it is possible to more effectively remove, from the composition, foreign substances having high polarity, which are likely to cause defects.

A pore diameter of the filter is preferably approximately 0.001 to 1.0 μm, more preferably approximately 0.02 to 0.5 μm, and still more preferably approximately 0.01 to 0.1 μm. In a case of setting the pore diameter of the filter within the above-described range, it is possible to reliably remove fine foreign substances contained in the composition while suppressing filtration clogging.

In a case of using a filter, different filters may be combined.

In addition, it is preferable that the filter to be used is treated before filtering the composition. A liquid used for the treatment is not particularly limited, but it is preferably the composition or the liquid containing components which are contained in the concentrated solution and the composition.

In a case of carrying out the filtering, an upper limit value of a temperature during the filtering is preferably room temperature (25° C.) or lower, more preferably 23° C. or lower, and still more preferably 20° C. or lower. In addition, a lower limit value of the temperature during the filtering is preferably 0° C. or higher, more preferably 5° C. or higher, and still more preferably 10° C. or higher.

In the filtering, particulate foreign substances and/or impurities can be removed. However, in a case where the filtering is carried out at the above-described temperature, the amount of the particulate foreign substances and/or impurities dissolved in the composition is reduced, and thus the filtering is carried out more efficiently.

### <Destaticization Step>

The above-described production method may further include a destaticization step of destaticizing at least one selected from the group consisting of the present composition, the concentrated solution, and the kit.

It is preferable that all the steps involved in the above-described production method are carried out in a clean room. It is preferable that the clean room satisfies 14644-1 clean room standards. It is preferable that the clean room satisfies any of International Organization for Standardization (ISO) class 1, ISO class 2, ISO class 3, or ISO class 4, it is more preferable to satisfy ISO class 1 or ISO class 2, and it is still more preferable to satisfy ISO class 1.

### <Container>

The container which accommodates the above-described present composition, concentrated solution, or kit is not particularly limited as long as corrosiveness due to the liquid does not cause a problem, and a well-known container can be used.

The container is preferably a container for a use application in a semiconductor, which has high internal cleanliness and hardly causes elution of impurities.

Specific examples of the above-described container include “CLEAN BOTTLE” series manufactured by AICELLO CHEMICAL Co., Ltd. and “PURE BOTTLE” manufactured by KODAMA PLASTICS Co., Ltd. In addition, for the intended purpose of preventing the mixing (contamination) of raw materials and impurities into the composition, it is also preferable to use a multi-layer container in which an interior wall of the container has a six-layer structure consisting of six kinds of resins and a multi-layer container in which an interior wall of the container has a seven-layer structure consisting of six kinds of resins. Examples of these containers include containers described in JP2015-123351A, but the container is not limited thereto.

In addition, as the container, containers exemplified in paragraphs to of WO2022/004217A can also be used, the contents of which are incorporated herein by reference.

It is preferable to wash the inside of these containers before being filled. A liquid used for washing may be appropriately selected according to the use application, but it is preferably the present composition, a liquid obtained by diluting the present composition, or a liquid containing at least one of the components which are added to the present composition.

In order to prevent a change in components in the composition during storage, the inside of the container may be replaced with inert gas (nitrogen, argon, or the like) with a purity of 99.99995% by volume or more. In particular, a gas having a low moisture content is preferable. Although the liquid container may be transported and stored at normal temperature, the temperature may be controlled in a range of −20° C. to 20° C. in order to prevent deterioration.

### [Substrate Treatment Method]

In a substrate treatment method using the present composition (hereinafter, also simply referred to as “present treatment method”), the present composition can be typically used by being brought into contact with an object to be treated containing a metal-containing substance which is a material containing a metal (particularly, a substrate having a metal-containing substance which is a material containing a metal) (hereinafter, also referred to as “object to be treated”). In this case, the object to be treated may contain a plurality of kinds of the metal-containing substances.

### [Object to be Treated]

The object to be treated, which is an object treated using the present composition, is preferably a substrate having a metal-containing substance.

“On the substrate” in the present specification includes, for example, any of front and back surfaces, side surfaces, an inside of a groove, and the like of the substrate. In addition, “metal-containing substance on the substrate” includes not only a case where the metal-containing substance is directly present on the surface of the substrate but also a case where the metal-containing substance is present on the substrate through another layer.

In addition, the “substrate” in the present specification includes, for example, a semiconductor substrate consisting of a single layer and a semiconductor substrate consisting of multiple layers.

The metal-containing substance is a material containing a simple body of a metal (metal atom) as a main component.

Examples of the metal contained in the metal-containing substance include at least one metal M selected from the group consisting of copper (Cu), cobalt (Co), tungsten (W), titanium (Ti), tantalum (Ta), ruthenium (Ru), chromium (Cr), hafnium (Hf), osmium (Os), platinum (Pt), nickel (Ni), manganese (Mn), zirconium (Zr), molybdenum (Mo), lanthanum (L^{a}), and iridium (Ir).

It is sufficient that the metal-containing substance is a substance containing a metal (metal atom), and examples thereof include a substance composed of at least one selected from the group consisting of a simple body of the metal M, an alloy containing the metal M, an oxide of the metal M, a nitride of the metal M, and an oxynitride of the metal M.

More specific examples of the metal-containing substance include a metal-containing substance containing at least one component selected from the group consisting of copper, cobalt, a cobalt alloy, tungsten, a tungsten alloy, ruthenium, a ruthenium alloy, tantalum, a tantalum alloy, aluminum oxide, aluminum nitride, aluminum oxynitride, titanium aluminum, titanium, titanium nitride, titanium oxide, zirconium oxide, hafnium oxide, tantalum oxide, lanthanum oxide, and a yttrium alloy.

In addition, the metal-containing substance may be a mixture containing two or more of these compounds.

A form of the metal-containing substance is not particularly limited, and it may be, for example, any of a film-shaped (layer-shaped) form, a wiring line-shaped form, and a particle-shaped form.

The metal-containing substance may be disposed only on one main surface of the substrate, or may be disposed on both main surfaces. In addition, the metal-containing substance may be disposed on the entire main surface of the substrate, or may be disposed on a part of the main surface of the substrate.

The substrate preferably has a metal M-containing substance containing the metal M; more preferably has a metal-containing substance containing at least one metal selected from the group consisting of W, Mo, Cu, Co, Ti, Ta, and Ru; still more preferably has a metal-containing substance containing at least one metal selected from the group consisting of W, Mo, Cu, and Co; and particularly preferably has a metal-containing substance containing at least one of W or a W alloy (W-containing substance).

Among these, the substrate preferably has a W-containing film, a Mo-containing film, a copper-containing film, a Co-containing film, or a Ti-containing film, and more preferably has a W-containing film or a Mo-containing film.

Examples of the W-containing film include a metal film consisting of only tungsten (a W metal film) and a metal film consisting of an alloy made of tungsten and another metal (a W alloy metal film). Specific examples of the W alloy metal film include a WTi alloy metal film and a WCo alloy metal film.

The tungsten-containing film is usually used as a wiring line film or a barrier metal.

Examples of the Mo-containing film include a metal film consisting of only molybdenum (an Mo metal film) and a metal film made of an alloy consisting of molybdenum and another metal (an Mo alloy metal film). Specific examples of the Mo alloy metal film include an MoCo alloy metal film.

Examples of the copper-containing film include a wiring line film consisting of only metallic copper (a copper wiring line film) and a wiring line film consisting of an alloy containing metallic copper and another metal (a copper alloy wiring line film).

Specific examples of the copper alloy wiring line film include a wiring line film consisting of an alloy of one or more metals selected from Al, Ti, Cr, Mn, Ta, and W, and copper. More specific examples of the copper alloy wiring line film include a CuAl alloy wiring line film, a CuTi alloy wiring line film, a CuCr alloy wiring line film, a CuMn alloy wiring line film, a CuTa alloy wiring line film, and a CuW alloy wiring line film.

Examples of the Co-containing film include a metal film consisting of only metallic cobalt (a Co metal film) and a metal film consisting of an alloy containing metallic cobalt and another metal (a Co alloy metal film).

Specific examples of the Co alloy metal film include a metal film consisting of an alloy of one or more metals selected from Ti, Cr, Fe, Ni, Mo, Pd, Ta, and W, and cobalt. More specific examples thereof include a CoTi alloy metal film, a CoCr alloy metal film, a CoFe alloy metal film, a CoNi alloy metal film, a CoMo alloy metal film, a CoPd alloy metal film, a CoTa alloy metal film, and a CoW alloy metal film.

Among the Co-containing films, the Co metal film is usually used as a wiring line film, and the Co alloy metal film is usually used as a barrier metal.

Examples of the Ti-containing film include a metal film containing an alloy consisting of Ti, Al, or the like, in which the metal film is a Ti alloy metal film which may further contain the above-described dopant. Specific examples of the Ti alloy metal film include a TiAl film, a TiAlC film, and a TiAlN film.

The Ti alloy metal film is usually used in a structure of a gate and a structure surrounding the gate.

More specific examples of the object to be treated include a laminate including, on a substrate, at least a metal layer, an insulating film, and a metal hard mask in this order. The laminate may further have holes formed from a surface (opening portion) of the metal hard mask toward the substrate so that a surface of the metal layer is exposed, as a result of undergoing a dry etching step or the like.

A manufacturing method of such a laminate having holes as described above is not particularly limited, and examples thereof include a method in which a laminate before treatment, including a substrate, a metal layer, an insulating film, and a metal hard mask in this order, is subjected to a dry etching step using the metal hard mask as a mask and an insulating film is etched so that the surface of the metal layer is exposed, thereby providing holes which penetrate through the metal hard mask and the inside of the insulating film.

A manufacturing method of the metal hard mask is not particularly limited, and for example, first, a metal layer containing a predetermined component is formed on an insulating film, and a resist film having a predetermined pattern is formed on the metal layer. Next, a method of manufacturing a metal hard mask (that is, a film in which the metal layer is patterned) by etching the metal layer using the resist film as a mask can be mentioned.

In addition, the laminate may include a layer other than the above-described layers, and examples thereof include an etching stop film and an antireflection film.

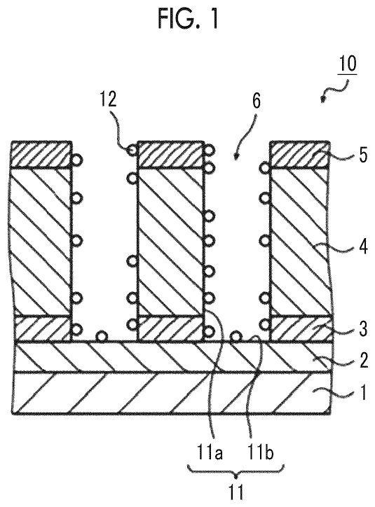

FIG. 1 shows a schematic cross-sectional view showing an example of the laminate which is the object to be treated in the present treatment method.

A laminate 10 shown in FIG. 1 includes a metal layer 2, an etching stop layer 3, an insulating film 4, and a metal hard mask 5 in this order on a substrate 1, and holes 6 by which the metal layer 2 is exposed is formed at a predetermined position as a result of a dry etching step or the like. That is, the object to be treated shown in FIG. 1 is a laminate which includes the substrate 1, the metal layer 2, the etching stop layer 3, the insulating film 4, and the metal hard mask 5 in this order, in which the holes 6 which penetrate from the surface of the metal hard mask 5 to the surface of the metal layer 2 at the position of the opening portion of the metal hard mask 5. An interior wall 11 of the holes 6 is consisting of a cross-sectional wall 11a consisting of the etching stop layer 3, the insulating film 4, and the metal hard mask 5, and a bottom wall 11b consisting of the exposed metal layer 2, and dry etching residues 12 are attached thereto.

The present treatment method can be suitably used for removing these dry etching residues 12. That is, while being excellent in the removability (residue removability) for the dry etching residues 12, it is also excellent in the corrosion prevention property with respect to the interior wall 11 (for example, the metal layer 2 and the like) of the object to be treated.

In addition, a laminate which has undergone a dry ashing step after the dry etching step may be subjected to the above-described substrate treatment method.

Hereinafter, each layer-constituting material of the above-described laminate will be described.

### <Metal Hard Mask>

The metal hard mask preferably contains at least one component selected from the group consisting of copper, cobalt, a cobalt alloy, tungsten, a tungsten alloy, ruthenium, a ruthenium alloy, tantalum, a tantalum alloy, aluminum oxide, aluminum nitride, aluminum oxynitride, titanium aluminum, titanium carbide aluminum, titanium, titanium nitride, titanium oxide, zirconium oxide, hafnium oxide, tantalum oxide, lanthanum oxide, and a yttrium alloy (preferably YSiOx). Here, x and y are preferably numbers represented by x=1 to 3 and y=1 to 2, respectively.

Examples of a material of the above-described metal hard mask include TiN, TiAl, TiAlC, WO₂, and ZrO₂.

### <Insulating Film>

A material of the insulating film is not particularly limited, and examples thereof include materials having a dielectric constant k of preferably 3.0 or less and more preferably 2.6 or less.

Specific examples of the material of the insulating film include organic polymers such as SiOx, SiN, SiOC, and polyimide. Here, x is preferably a number represented by 1 to 3.

The insulating film may be composed of a plurality of films. Examples of the insulating film composed of a plurality of films include an insulating film obtained by combining a film containing silicon oxide and a film containing oxidized silicon carbide.

### <Etching Stop Layer>

A material of the etching stop layer is not particularly limited. Specific examples of the material of the etching stop layer include materials based on SiN, SiON, and SiOCN, and metal oxides such as AlOx.

### <Metal Layer>

A material which forms the metal layer, which is a wiring line material and/or a plug material, is not particularly limited, but it preferably contains one or more selected from the group consisting of cobalt, tungsten, and copper. In addition, the material which forms the metal layer may be cobalt, tungsten, or an alloy of copper and another metal.

The metal layer may further contain a metal other than cobalt, tungsten, and copper, a metal nitride, and/or an alloy. Examples of the metal other than cobalt, tungsten, and copper, which may be contained in the metal layer, include titanium, titanium-tungsten, titanium nitride, tantalum, a tantalum compound, chromium, a chromium oxide, and aluminum.

The metal layer may contain at least one dopant selected from the group consisting of carbon, nitrogen, boron, and phosphorus, in addition to one or more selected from the group consisting of cobalt, tungsten, and copper.

### <Substrate>

Specific examples of a wafer constituting the substrate include a wafer consisting of silicon-based material, such as a silicon (Si) wafer, a silicon carbide (SiC) wafer, and a wafer based on a resin containing silicon (a glass epoxy wafer), a gallium phosphide (GaP) wafer, a gallium arsenide (GaAs) wafer, and an indium phosphide (InP) wafer.

The silicon wafer may be an n-type silicon wafer obtained by doping a silicon wafer with a pentavalent atom (for example, phosphorus (P), arsenic (As), or antimony (Sb)), or a p-type silicon wafer obtained by doping a silicon wafer with a trivalent atom (for example, boron (B) or gallium (Ga)). The silicon of the silicon wafer may be, for example, any of amorphous silicon, single crystal silicon, polycrystalline silicon, or polysilicon.

In addition to those described above, the above-described object to be treated may include various layers and/or structures as desired. For example, the substrate may include a metal wire, a gate electrode, a source electrode, a drain electrode, an insulating layer, a ferromagnetic layer, and/or a non-magnetic layer.

The substrate may include an exposed integrated circuit structure, for example, an interconnect mechanism such as a metal wire and a dielectric material. Examples of the metal and the alloy which are used in the interconnect mechanism include aluminum, a copper-aluminum alloy, copper, titanium, tantalum, cobalt, silicon, titanium nitride, tantalum nitride, and tungsten. The substrate may include layers of silicon oxide, silicon nitride, silicon carbide, and/or silicon oxide dope with carbon.

The manufacturing method of the object to be treated is not particularly limited as long as it is a method which is generally carried out in this field.

Examples of a method of forming the insulating film on the wafer constituting the substrate include a method in which a wafer constituting a substrate is subjected to a heat treatment in the presence of an oxygen gas to form a silicon oxide film, and then a gas of silane and ammonia is introduced thereto to form a silicon nitride film by a chemical vapor deposition (CVD) method.

Examples of a method of forming the above-described metal-containing layer on the wafer constituting the substrate include a method in which a circuit is formed on the wafer having an insulating film by a well-known method such as resist, and then a metal-containing layer is formed by a method such as plating, a sputtering method, a CVD method, or a molecular beam epitaxy (MBE) method.

The object to be treated may be a substrate which has been subjected to a flattening treatment such as a CMP treatment after providing an insulating film, a barrier metal, and a metal-containing film on the wafer. The CMP treatment is a treatment of flattening a surface of a substrate having a metal-containing film, a barrier metal, and an insulating film by a combined action of chemical action using a polishing slurry containing polishing fine particles (abrasive grains) and mechanical polishing.

The surface of the substrate which has been subjected to the CMP treatment may have impurities remaining thereon, such as abrasive grains (for example, silica and alumina) used in the CMP treatment, a polished metal-containing film, and metal impurities (metal residue) derived from the barrier metal. For example, since these impurities may short-circuit the wiring lines and deteriorate the electrical characteristics of the substrate, the substrate that has been subjected to the CMP treatment is subjected to a washing treatment for removing these impurities from the surface.

Specific examples of the substrate which has been subjected to the CMP treatment include the substrate that has been subjected to a CMP treatment, described in Vol. 84, No. 3, 2018, but the present invention is not limited thereto.

### [Step A]

Examples of the present treatment method include a treatment method including a step A of bringing the present composition into contact with the substrate having a metal-containing substance. By performing the present step A, the metal-containing substance on the substrate can be removed.

The present composition used in the step A is as described above.

In addition, the substrate having a metal-containing substance, which is the object to be treated in the step A, is as described above. The substrate having a metal-containing substance is preferably a substrate having a W-containing substance or a substrate having a Mo-containing substance.

The method of bringing the present composition into contact with the object to be treated (substrate having a metal-containing substance) is not particularly limited, and examples thereof include a method of immersing the object to be treated in the present composition contained in a tank, a method of spraying the present composition onto the substrate, a method of flowing the present composition onto the substrate, and any combination thereof. Among these, a method of immersing, in the present composition, the substrate having a metal-containing substance, which is the object to be treated, is preferable.

In order to further enhance treatment ability of the present composition, a mechanical stirring method may also be used.

Examples of the mechanical stirring method include a method of circulating the present composition on the substrate, a method of flowing or spraying the present composition onto the substrate, and a method of stirring the present composition with an ultrasonic wave or a megasonic wave.

In addition, the treatment by immersion may be a batch method in which a plurality of objects to be treated are immersed in a treatment tank or may be a single substrate method.

A treatment time in the step A may be adjusted according to the method of bringing the present composition into contact with the substrate, the temperature of the present composition, and the like. The treatment time (time for contact between the composition and the object to be treated) is not particularly limited, but is preferably 0.25 to 10 minutes and more preferably 0.5 to 2 minutes.

The temperature of the present composition at the time of the treatment is not particularly limited, but the lower limit thereof is preferably 15° C. or higher, more preferably 20° C. or higher, and still more preferably 30° C. or higher. In addition, the upper limit thereof is preferably 90° C. or lower, more preferably 80° C. or lower, and still more preferably 70° C. or lower.

Examples of a specific aspect of the step A include a step A1 of subjecting a wiring line consisting of the metal-containing substance, disposed on the substrate, to a recess-etching treatment using the present composition; a step A2 of removing a film on an outer edge of the substrate on which a film consisting of the metal-containing substance is disposed, using the present composition; a step A3 of removing the metal-containing substance which has adhered to a back surface of the substrate on which a film consisting of the metal-containing substance is disposed, using the present composition; a step A4 of removing the metal-containing substance on the substrate after dry etching, using the present composition; and a step A5 of removing the metal-containing substance on the substrate after the chemical-mechanical polishing treatment, using the present composition.

For the above-described steps A1 to A5, the description in paragraphs to of WO2019/138814A, the contents of which are incorporated in the present specification by reference, can be referenced.

### [Rinsing Step]

The present treatment method may further include a step (hereinafter, referred to as “step B”) of performing a rinsing treatment (rinsing and cleaning with a solvent) on the object to be treated (substrate having a metal-containing substance) using a rinsing liquid, after the step A.

The step B is performed continuously after the step A, and it is preferably a step of performing the rinsing treatment with a rinsing liquid for 5 seconds to 5 minutes. The step B may be performed by the above-described mechanical stirring method.

Examples of a solvent of the rinsing liquid include deionized (DI) water, methanol, ethanol, isopropanol, N-methylpyrrolidinone, γ-butyrolactone, dimethyl sulfoxide, ethyl lactate, and propylene glycol monomethyl ether acetate.

The solvent of the rinsing liquid is preferably DI water, methanol, ethanol, isopropanol, or a mixed liquid thereof, and more preferably DI water, isopropanol, or a mixed liquid of DI water and isopropanol.

As a method of bringing the rinsing liquid into contact with the object to be treated, the above-described method of bringing the present composition into contact with the object to be treated can be similarly applied.

A temperature of the rinsing solvent in the step B is preferably 16° C. to 27° C.

### [Drying Step]

The present treatment method may include a step C of drying the object to be treated (the substrate having a metal-containing substance), after the step B.

The drying method is not particularly limited, and examples thereof include a spin drying method, a method of flowing a dry gas onto the object to be treated, a method of heating the substrate by a heating unit such as a hot plate and an infrared lamp, a Marangoni drying method, a Rotagoni drying method, an isopropanol (IPA) drying method, and any combinations thereof.

A drying time in the step C depends on the drying method, but it is preferably 20 seconds to 5 minutes.

In the step C, it is preferable to dry the substrate by heating the substrate with a heating unit, from the viewpoint of excellent removability of the composition in the SiOx layer.

In this case, a heating temperature is not particularly limited, but it is preferably 50° C. to 350° C. and more preferably higher than 100° C. and lower than 400° C. from the viewpoint of being more excellent in balance between the removability of the composition in the SiOx layer and the film reduction in the Co film and the SiOx layer, and it is still more preferably 150° C. to 250° C. from the viewpoint of being more excellent in removability of the composition in the Co film and the SiOx layer.

### [Manufacturing Method of Semiconductor Device]

The present invention also includes a manufacturing method of a semiconductor device. The manufacturing method of a semiconductor device according to the embodiment of the present invention preferably includes a substrate treatment method including the above-described step A.

### [Compound]

The present invention also includes a compound. The compound according to the embodiment of the present invention is the above-described compound represented by Formula (F).

### EXAMPLES

Hereinafter, the present invention will be described in more detail with reference to Examples.

The materials, the amounts of materials used, the proportions, the treatment details, the treatment procedure, and the like shown in Examples below may be modified as appropriate as long as the modifications do not depart from the spirit of the present invention. Therefore, the scope of the present invention should not be construed as being limited to Examples shown below.

### Synthesis of Specific Resin E-1 (Synthesis Example 1)

A specific resin E-1 was synthesized according to the following scheme.

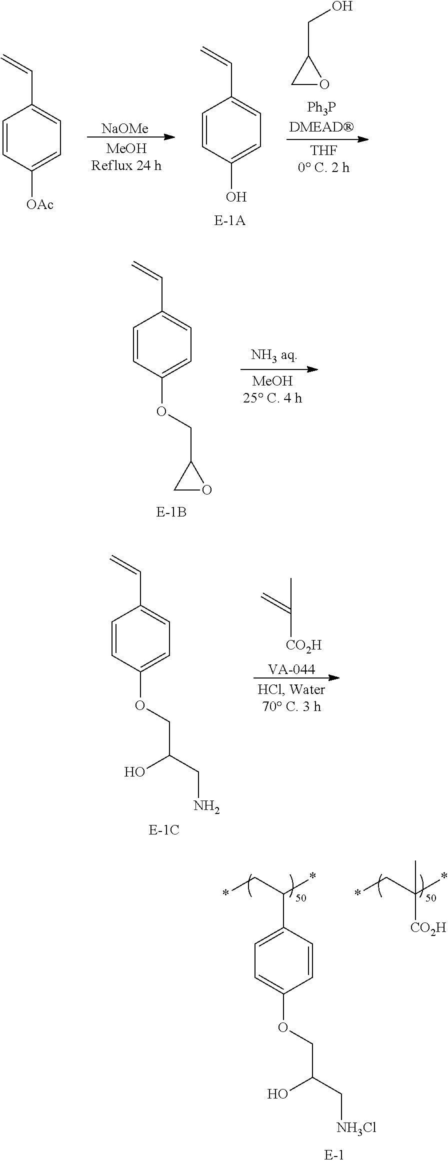

### Synthesis of Intermediate E-1A

4-Acetoxystyrene (50.0 g, 0.3 mol, manufactured by Tokyo Chemical Industry Co., Ltd.) and methanol (500 mL, manufactured by FUJIFILM Wako Pure Chemical Corporation) were charged into a 1.0 L three-neck flask under a nitrogen flow (50 mL/min), and the obtained reaction solution was stirred at 25° C. Thereafter, sodium methoxide (48.6 g, 0.9 mol, manufactured by FUJIFILM Wako Pure Chemical Corporation) was added to the reaction solution, and the obtained reaction solution was heated under reflux for 24 hours.

Next, the solvent was distilled off from the obtained reaction solution under reduced pressure of 40° C./10 hPa. tert-Butyl methyl ether (300 mL, manufactured by FUJIFILM Wako Pure Chemical Corporation) and 3M hydrochloric acid (400 mL) were added to the obtained crude product, and the obtained solution was transferred to a 1 L separating funnel, and stirred. After stirring, a lower phase (water phase) was removed and an upper phase (organic phase) was recovered. Distilled water (300 mL) was added to the obtained organic phase and stirred, the solution obtained after the stirring was allowed to stand to remove the lower phase (water phase), and the upper phase (organic phase) was recovered. Distilled water (300 mL) was added to the obtained organic phase and stirred, the solution obtained after the stirring was allowed to stand to remove the lower phase (water phase), and the upper phase (organic phase) was recovered. Thereafter, the solvent was distilled off from the obtained organic phase under reduced pressure of 40° C./10 hPa to obtain an intermediate E-1A.

### Synthesis of Intermediate E-1B

The intermediate E-1A (35.0 g, 0.29 mol), triphenylphosphine (98.8 g, 0.38 mol, manufactured by FUJIFILM Wako Pure Chemical Corporation), glycidol (28.2 g, 0.38 mol, manufactured by Tokyo Chemical Industry Co., Ltd.), and tetrahydrofuran (THF; 245 mL, manufactured by FUJIFILM Wako Pure Chemical Corporation) were charged into a 0.5 L three-neck flask under a nitrogen flow (50 mL/min), and the obtained reaction solution was cooled to 0° C.

Next, a dissolving solution obtained by dissolving bis(2-methoxyethyl) azodicarboxylate (DMEAD (registered trademark), 88.9 g, 0.38 mol, manufactured by FUJIFILM Wako Pure Chemical Corporation) in THF (105 mL) was separately prepared.

While maintaining the internal temperature of the reaction solution obtained above at 5° C. or lower, the above-described dissolving solution was added dropwise to the reaction solution over 2 hours. After completion of the dropwise addition, the obtained reaction solution was stirred at 0° C. for 2 hours.

Next, the solvent was distilled off from the obtained reaction solution under reduced pressure of 40° C./10 hPa. tert-Butyl methyl ether (300 mL, manufactured by FUJIFILM Wako Pure Chemical Corporation) and distilled water (400 mL) were added to the obtained crude product, and the obtained solution was transferred to a 1 L separating funnel, and stirred. After stirring, a lower phase (water phase) was removed and an upper phase (organic phase) was recovered. Distilled water (400 mL) was added to the obtained organic phase and stirred, the solution obtained after the stirring was allowed to stand to remove the lower phase (water phase), and the upper phase (organic phase) was recovered. The solvent was distilled off from the obtained organic phase under reduced pressure of 40° C./10 hPa.

The obtained crude product was purified by silica gel column chromatography to obtain an intermediate E-1B.

### Synthesis of Intermediate E-1C

E-1B (60.0 g, 0.3 mol) and methanol (1.5 L, manufactured by FUJIFILM Wako Pure Chemical Corporation) were charged into a 5.0 L three-neck flask, and the obtained reaction solution was stirred at 25° C. Thereafter, 28% aqueous ammonia (1.8 L, manufactured by FUJIFILM Wako Pure Chemical Corporation) was added to the reaction solution, and the obtained reaction solution was stirred at 25° C. for 4 hours.

The obtained reaction solution was cooled to 0° C., and a precipitate (precipitate 1) was filtered. 6.0 L of distilled water was added to the obtained filtrate while maintaining the temperature at 0° C., and a precipitate (precipitate 2) was filtered. The obtained precipitate 1 and precipitate 2 were combined and washed twice with 0.5 L of distilled water. Thereafter, blast drying was performed at 40° C. to obtain an intermediate E-1C.

The ¹H-nuclear magnetic resonance (NMR) data of the obtained intermediate E-1C are shown below.

¹H-NMR (400 MHZ, DMSO-d6): δ (ppm)=7.38 (d, J=8.7 Hz, 2H), 6.90 (d, J=8.7 Hz, 2H), 6.65 (dd, J=10.9 Hz, 17.7 Hz, 1H), 5.67 (d, J=17.6 Hz, 1H), 5.10 (d, J=10.9 Hz, 1H), 3.60 to 3.98 (m, 3H), 2.52 to 2.72 (m, 2H)

### Synthesis of Specific Resin E-1

The intermediate E-1C (25.1 g, 0.13 mol) and distilled water (120 g) were charged into a 0.5 L three-neck flask under a nitrogen flow (50 mL/min), and the mixture was cooled to 0° C. 1 M hydrochloric acid (130 mL) and methacrylic acid (11.2 g, 0.13 mol, manufactured by FUJIFILM Wako Pure Chemical Corporation) were added to the obtained reaction solution, and the obtained reaction solution was stirred at 70° C. for 30 minutes.

Next, a dissolving solution obtained by dissolving VA-044 (2.1 g, 6.5 mmol, manufactured by FUJIFILM Wako Pure Chemical Corporation) in distilled water (10.5 mL) was separately prepared.

The above-described dissolving solution was added to the reaction solution obtained above, and the obtained reaction solution was further stirred at 70° C. for 3 hours.

After confirming that signals derived from the intermediate E-1C and the methacrylic acid had disappeared by ¹H-NMR, the resultant was subjected to a filtration to remove dust, thereby obtaining an aqueous solution containing a specific resin E-1 (concentration of solid contents: 13.1% by mass).

The ¹H-NMR data of the obtained specific resin E-1 is shown below.

¹H-NMR (400 MHZ, D₂O): δ (ppm)=6.50 to 8.00 (broad, 4H), 4.08 to 4.24 (broad, 1H), 3.86 to 4.08 (broad, 3H), 3.12 to 3.24 (broad, 1H), 2.94 to 3.12 (broad, 1H), 0.70 to 2.90 (broad, 7H)

### Synthesis of Specific Resin E-16 (Synthesis Example 2)

A specific resin E-16 was synthesized according to the following scheme.

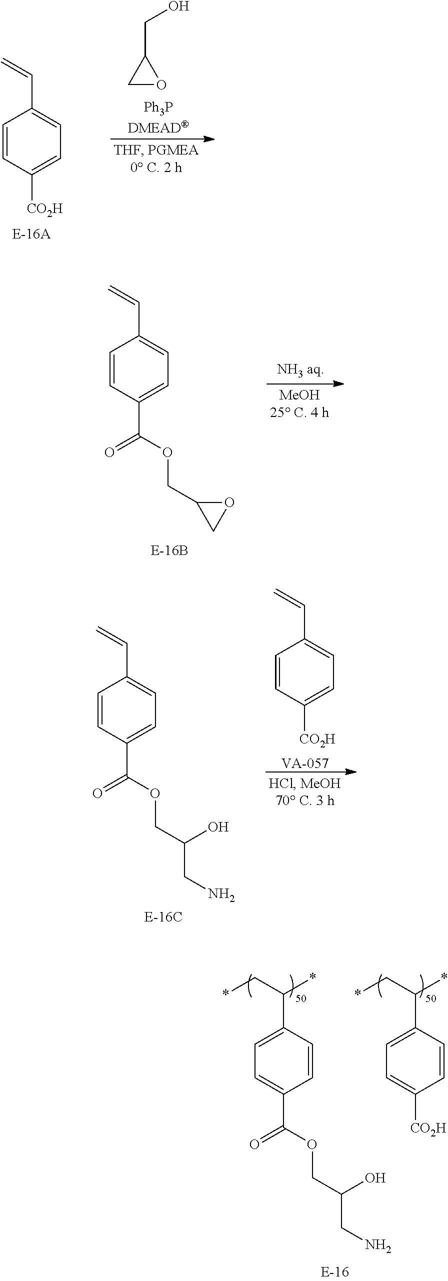

### Synthesis of Intermediate E-16C

An intermediate E-16C was synthesized according to the procedure described in [Synthesis of intermediate E-1B] and [Synthesis of intermediate E-1C] above, except that, in [Synthesis of intermediate E-1B] described above, E-16A was used instead of E-1A.

The ¹H-NMR data of the obtained intermediate E-16C is shown below.

¹H-NMR (400 MHZ, DMSO-d6): δ (ppm)=8.00 (d, J=8.7 Hz, 2H), 7.47 (d, J=8.7 Hz, 2H), 6.76 (dd, J=10.9 Hz, 17.7 Hz, 1H), 5.89 (d, J=17.6 Hz, 1H), 5.60 (d, J=10.9 Hz, 1H), 3.80 to 4.18 (m, 3H), 2.72 to 2.92 (m, 2H)

### Synthesis of Specific Resin E-16

A specific resin E-16 was synthesized according to the procedure described in [Synthesis of specific resin E-1] above, except that, in [Synthesis of specific resin E-1] described above, E-16C was used instead of E-1C and 4-carboxystyrene was used instead of methacrylic acid.

The ¹H-NMR data of the obtained specific resin E-16 is shown below.

¹H-NMR (400 MHZ, D₂O): δ (ppm)=6.50 to 8.00 (broad, 8H), 4.28 to 4.44 (broad, 1H), 4.06 to 4.28 (broad, 4H), 3.22 to 3.44 (broad, 1H), 2.94 to 3.12 (broad, 1H), 0.70 to 2.90 (broad, 4H)

### Synthesis of Specific Resin E-17 (Synthesis Example 3)

A specific resin E-17 was synthesized according to the following scheme.

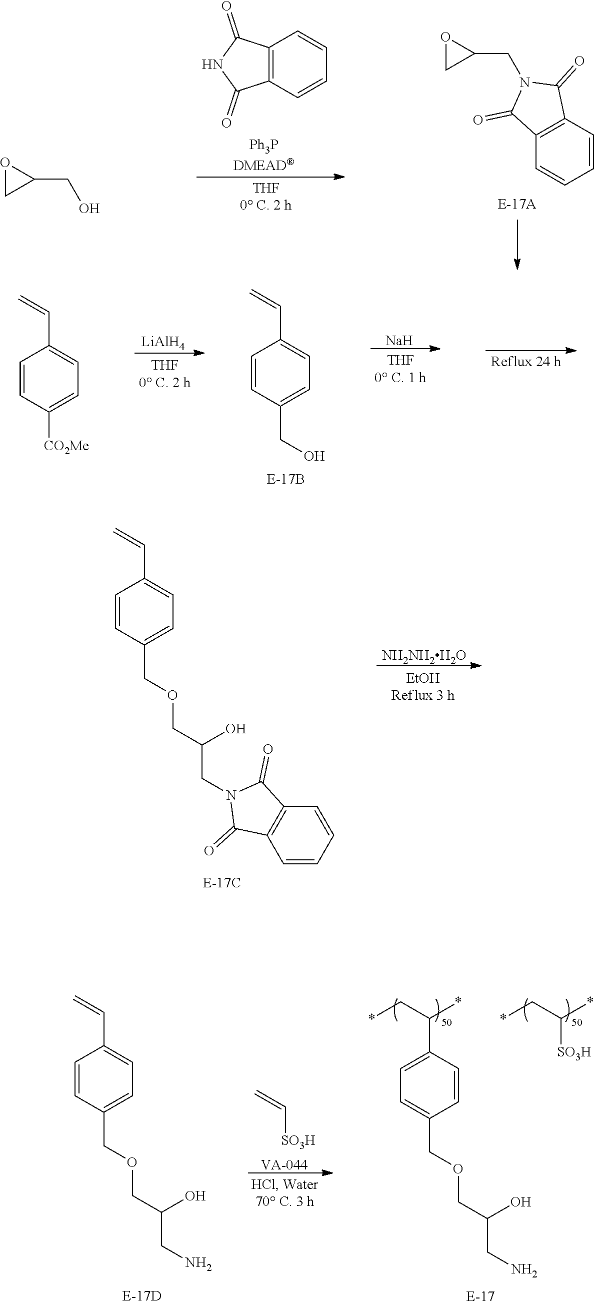

### Synthesis of Intermediate E-17A

Glycidol (37.0 g, 0.5 mol), triphenylphosphine (157.2 g, 0.6 mol, manufactured by FUJIFILM Wako Pure Chemical Corporation), phthalimide (88.2 g, 0.6 mol, manufactured by Tokyo Chemical Industry Co., Ltd.), and THF (259 mL, manufactured by FUJIFILM Wako Pure Chemical Corporation) were charged into a 0.5 L three-neck flask under a nitrogen flow (50 mL/min), and the obtained mixed solution was cooled to 0° C.

Next, a dissolving solution obtained by dissolving DMEAD (registered trademark) (40.4 g, 0.6 mol, manufactured by FUJIFILM Wako Pure Chemical Corporation) in THF (111 mL) was separately prepared.

While maintaining the internal temperature of the mixed solution obtained above at 5° C. or lower, the above-described dissolving solution was added dropwise to the mixed solution over 2 hours. After completion of the dropwise addition, the obtained reaction solution was stirred at 0° C. for 2 hours.

Next, the solvent was distilled off from the obtained reaction solution under reduced pressure of 40° C./10 hPa. tert-Butyl methyl ether (300 mL, manufactured by FUJIFILM Wako Pure Chemical Corporation) and distilled water (400 mL) were added to the obtained crude product, and the obtained solution was transferred to a 1 L separating funnel, and stirred. After stirring, a lower phase (water phase) was removed and an upper phase (organic phase) was recovered. Distilled water (400 mL) was added to the obtained organic phase and stirred, the solution obtained after the stirring was allowed to stand to remove the lower phase (water phase), and the upper phase (organic phase) was recovered. The solvent was distilled off from the obtained organic phase under reduced pressure of 40° C./10 hPa.

The obtained crude product was purified by silica gel column chromatography to obtain an intermediate E-17A.

### Synthesis of Intermediate E-17B

An intermediate E-17B was synthesized according to a method described in J. Org. Chem. 2002, 67, 5838 using methyl 4-vinylbenzoate as a starting material.

### Synthesis of Intermediate E-17C

The intermediate E-17B (13.4 g, 0.1 mol) and THF (400 mL, manufactured by FUJIFILM Wako Pure Chemical Corporation) were charged into a 0.5 L three-neck flask under a nitrogen flow (50 mL/min), and the mixture was cooled to 0° C. Thereafter, 60% NaH (4.0 g, 0.1 mol) was added thereto little by little, and the mixture was further stirred at 0° C. for 1 hour. Thereafter, the intermediate E-17A (20.3 g, 0.1 mol) was added thereto, and the obtained reaction solution was heated under reflux for 24 hours.

The reaction solution was cooled to 0° C., water was added thereto to stop the reaction, and the solvent was distilled off from the obtained reaction solution under reduced pressure of 40° C./10 hPa. tert-Butyl methyl ether (300 mL, manufactured by FUJIFILM Wako Pure Chemical Corporation) and distilled water (400 mL) were added to the obtained crude product, and the obtained solution was transferred to a 1 L separating funnel, and stirred. After stirring, a lower phase (water phase) was removed and an upper phase (organic phase) was recovered. Distilled water (400 mL) was added to the obtained organic phase and stirred, the solution obtained after the stirring was allowed to stand to remove the lower phase (water phase), and the upper phase (organic phase) was recovered. The solvent was distilled off from the obtained organic phase under reduced pressure of 40° C./10 hPa.

The obtained crude product was purified by silica gel column chromatography to obtain an intermediate E-17C.

### Synthesis of Intermediate E-17D

The intermediate E-17C (27.0 g, 0.08 mol) and ethanol (250 mL, manufactured by FUJIFILM Wako Pure Chemical Corporation) were charged into a 1.0 L three-neck flask under a nitrogen flow (50 mL/min), and stirred. Hydrazine monohydrate (23.3 g, 0.47 mmol, manufactured by FUJIFILM Wako Pure Chemical Corporation) was further added to the obtained reaction solution, and the reaction solution was heated under reflux for 3 hours. Thereafter, the obtained reaction solution was cooled to 0° C., and the precipitate was filtered, and the collected product was washed with THF (60 mL) cooled to 0° C., and the washing solution was recovered. The obtained filtrate and the recovered washing solution were mixed, the mixed solution was concentrated, dichloromethane (300 mL, manufactured by FUJIFILM Wako Pure Chemical Corporation) and a 20% by mass sodium hydroxide aqueous solution (300 mL) were added to the obtained crude product, and the obtained solution was transferred to a 1 L separating funnel and stirred. After stirring, a lower phase (water phase) was removed and an upper phase (organic phase) was recovered. The obtained organic phase was concentrated to obtain an intermediate E-17D.

The ¹H-NMR data of the obtained intermediate E-17D is shown below.

¹H-NMR (400 MHz, DMSO-d6): δ (ppm)=7.38 (d, J=8.7 Hz, 2H), 6.90 (d, J=8.7 Hz, 2H), 6.65 (dd, J=10.9 Hz, 17.7 Hz, 1H), 5.67 (d, J=17.6 Hz, 1H), 5.10 (d, J=10.9 Hz, 1H), 4.42 (s, 2H), 3.60 to 3.98 (m, 3H), 2.52 to 2.72 (m, 2H)

### Synthesis of Specific Resin E-17

A specific resin E-17 was synthesized according to the procedure described in [Synthesis of specific resin E-1] above, except that, in [Synthesis of specific resin E-1] described above, E-17D was used instead of E-1C and vinylsulfonic acid was used instead of methacrylic acid.

The ¹H-NMR data of the obtained specific resin E-17 is shown below.

¹H-NMR (400 MHZ, D₂O): δ (ppm)=6.50 to 8.00 (broad, 4H), 4.08 to 4.24 (broad, 3H), 3.86 to 4.08 (broad, 3H), 3.12 to 3.24 (broad, 1H), 2.94 to 3.12 (broad, 1H), 0.70 to 2.90 (broad, 4H)

### Synthesis of Specific Resin E-18 (Synthesis Example 4)

A specific resin E-18 was synthesized according to the following scheme.

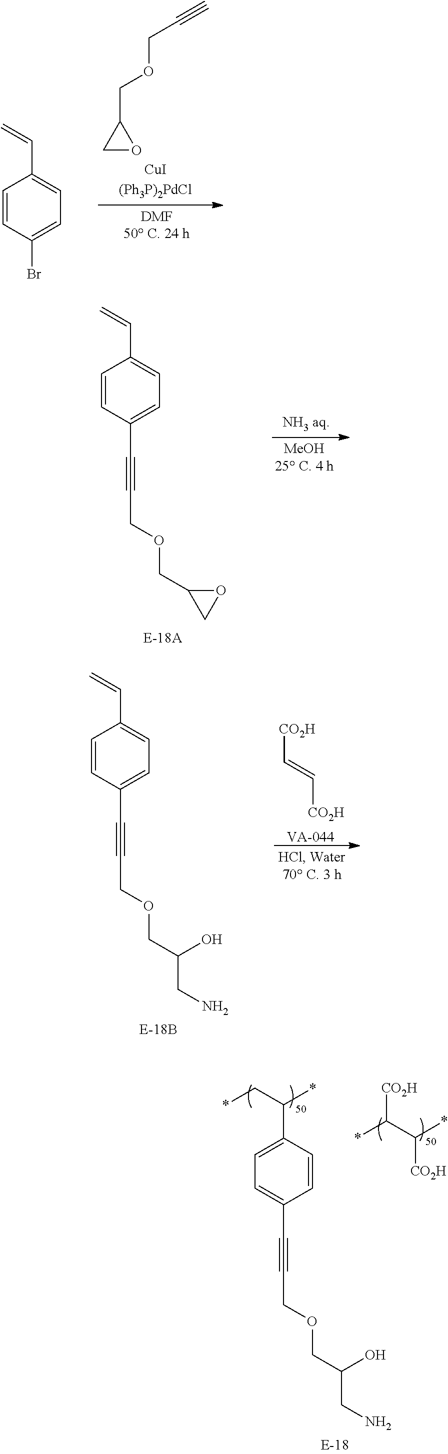

### Synthesis of Intermediate E-18A

An intermediate E-18A was synthesized according to a method described in Org. Lett. 2000, 2, 1729 using 4-bromostyrene as a starting material.

### Synthesis of Intermediate E-18B

An intermediate E-18B was synthesized according to the procedure described in [Synthesis of intermediate E-1C] above, except that E-18A was used instead of the intermediate E-1B.

The ¹H-NMR data of the obtained intermediate E-18B is shown below.

¹H-NMR (400 MHZ, DMSO-d6): δ (ppm)=8.00 (d, J=8.7 Hz, 2H), 7.47 (d, J=8.7 Hz, 2H), 6.76 (dd, J=10.9 Hz, 17.7 Hz, 1H), 5.89 (d, J=17.6 Hz, 1H), 5.60 (d, J=10.9 Hz, 1H), 4.40 (s, 2H), 3.80 to 4.18 (m, 3H), 2.72 to 2.92 (m, 2H)

### Synthesis of Specific Resin E-18

A specific resin E-18 was synthesized according to the procedure described in [Synthesis of specific resin E-1] above, except that, in [Synthesis of specific resin E-1] described above, E-18B was used instead of E-1C and maleic acid was used instead of methacrylic acid.

The ¹H-NMR data of the obtained specific resin E-18 is shown below.

¹H-NMR (400 MHZ, D₂O): δ (ppm)=6.50 to 8.00 (broad, 4H), 4.08 to 4.24 (broad, 3H), 3.86 to 4.08 (broad, 3H), 3.12 to 3.24 (broad, 1H), 2.94 to 3.12 (broad, 3H), 0.70 to 2.90 (broad, 2H)

### Raw Material of Composition

The following compounds were used to prepare a composition. As the various components used in Example, those all classified into a semiconductor grade or a high-purity grade equivalent thereto were used.

### Specific Resin and Other Resins

The specific resins E-1 to E-19 and resins CE-1 and CE-2 (resins not corresponding to the specific resins) were used.

In the following structural formulae, the repeating unit in the leftmost column corresponds to the repeating unit A, and the repeating unit in the rightmost column corresponds to the repeating unit B. However, in the specific resin E-7, the repeating unit in the center column corresponds to the repeating unit A, and the repeating unit in the rightmost column corresponds to the repeating unit B.

In the following structural formulae, “Mw” is a weight-average molecular weight of each resin measured by the above-described method. In addition, numbers appended to the repeating units represent the compositional ratios (molar fractions) of the respective repeating units in the resin. The compositional ratio of each repeating unit in the resin was measured by ¹³C-NMR.

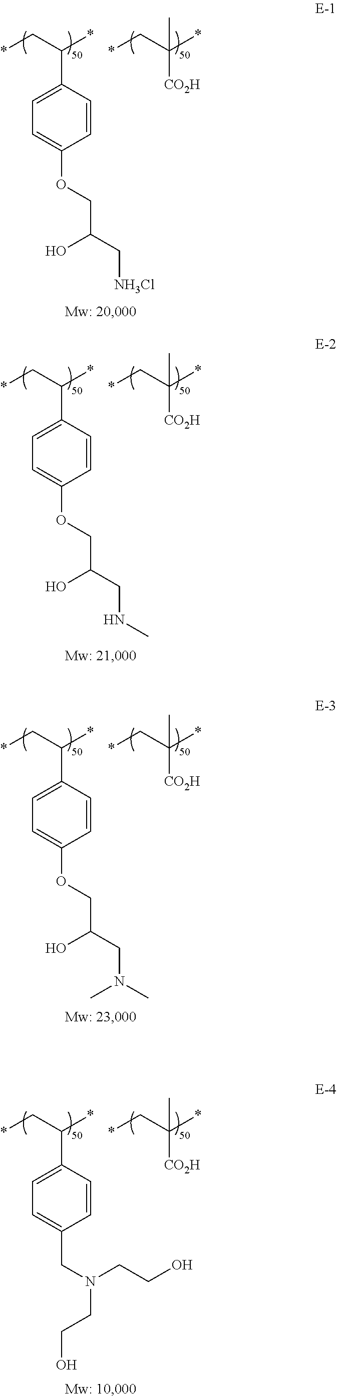

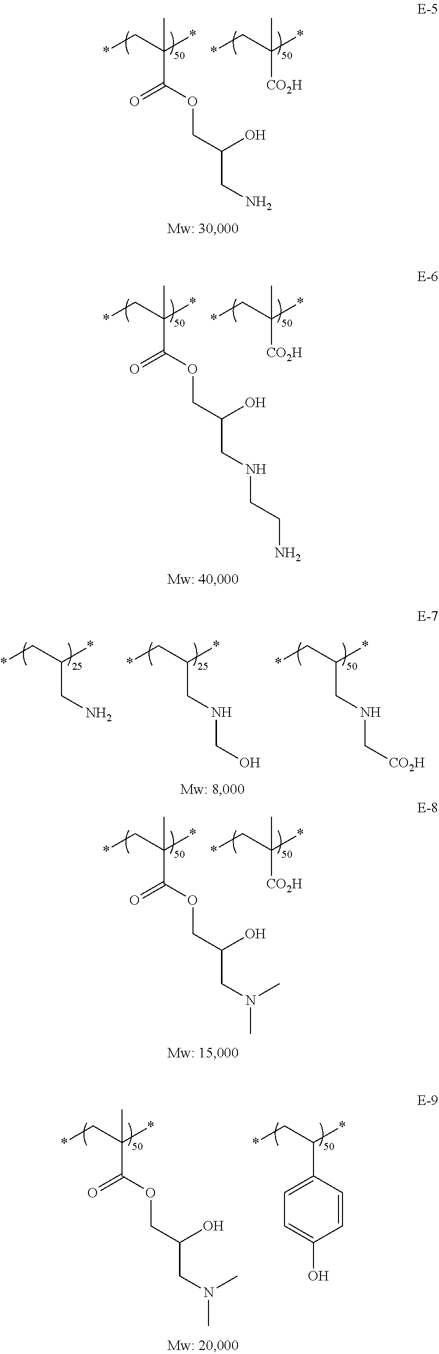

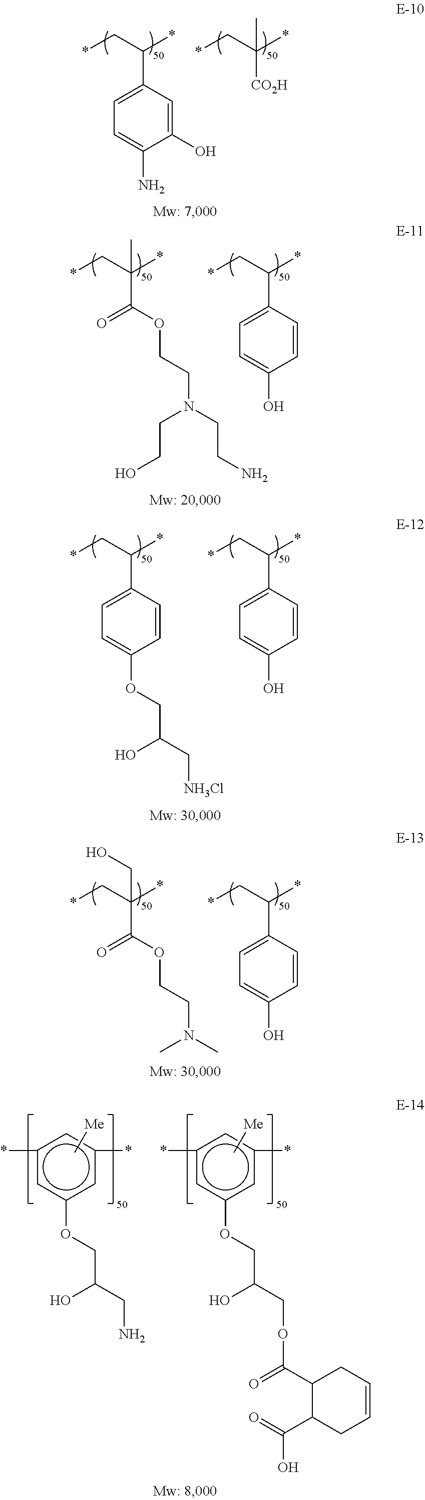

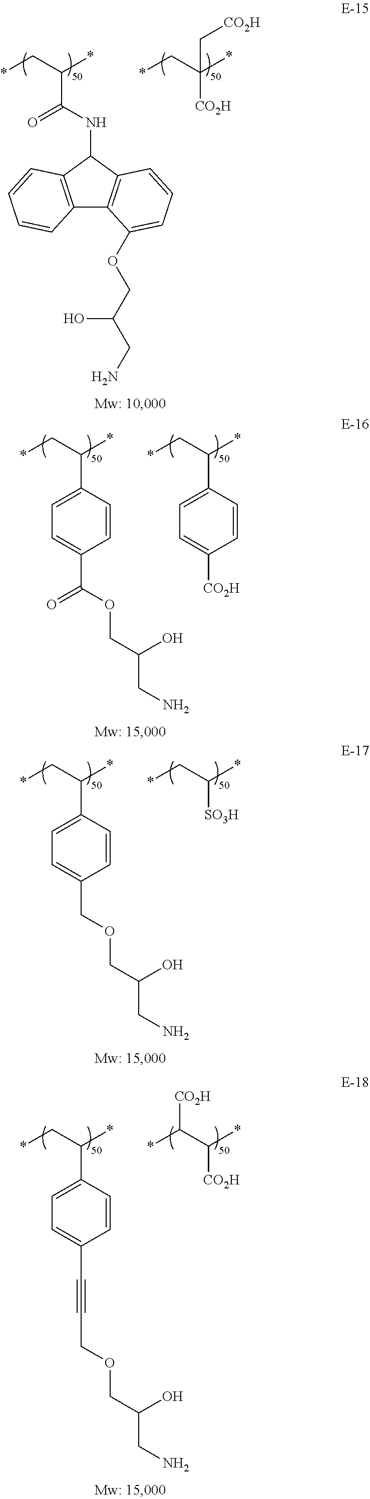

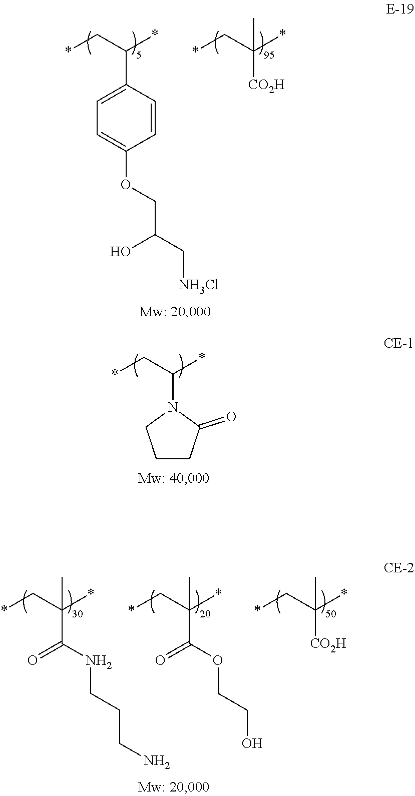

### <Removing Agent>

Hydroxylamine (corresponding to a hydroxylamine compound)
Ammonium hydroxide (corresponding to a basic compound)
Tetramethylammonium hydroxide (TMAH) (corresponding to a basic compound)
Hydrofluoric acid (corresponding to a fluorine-containing compound)
Sulfuric acid (corresponding to an inorganic acid)
Citric acid (corresponding to an organic acid)
Tartaric acid (corresponding to an organic acid)
Succinic acid (corresponding to an organic acid)
Ethylenediaminetetraacetic acid (EDTA) (corresponding to an organic acid)
Trishydroxymethylaminomethane (Tris) (corresponding to a basic compound)
Monoethanolamine (corresponding to a basic compound)
Tris(2-hydroxyethyl)methylammonium hydroxide (corresponding to a basic compound)
2-Dimethylamino-2-methyl-1-propanol (corresponding to a basic compound)

### <Oxidizing Agent>

Hydrogen peroxide

### <Corrosion Inhibitor>

5-Methyl-1H-benzotriazole
5-Mercapto-1-phenyl-1H-tetrazole (corresponding to a tetrazole compound)
n-Dodecyl mercaptan (corresponding to a thiol compound)

### <Surfactant>

PERSOFT (registered trademark) SF-T (an alkyl sulfate ester triethanolamine salt, anionic surfactant, manufactured by NOF CORPORATION)
NISSAN CATION (registered trademark) BB (dodecyltrimethylammonium chloride, cationic surfactant, manufactured by NOF CORPORATION)
NONION K-220 (polyoxyethylene lauryl ether, nonionic surfactant, manufactured by NOF CORPORATION)
NISSAN ANON (registered trademark) BL (betaine lauryldimethylaminoacetate, amphoteric surfactant, manufactured by NOF CORPORATION)

### <Antifoaming Agent>

KF-6701 (silicone-based antifoaming agent, manufactured by Shin-Etsu Chemical Co., Ltd.)

### <Solvent>

Propylene glycol
Water (ultrapure water)

### <pH Adjusting Agent>

Citric acid
Monoethanolamine
Nitric acid (HNO₃)
Potassium hydroxide (KOH)

### Example A

### Preparation of Composition

A preparation method for each of compositions of Examples A1 to A21 and Comparative Examples A1 and A2 will be described with reference to Example A1.

Hydroxylamine, 5-methyl-1H-benzotriazole, PERSOFT SF-T, and ultrapure water were mixed such that amounts thereof were the contents shown in Table 2 described later, whereby a mixed solution C-1 was obtained.

Thereafter, the mixed solution C-1, the specific resin E-1, and at least one of citric acid or monoethanolamine as a pH adjusting agent were sufficiently stirred using a stirrer to prepare a composition (washing treatment liquid) of Example A1.

The contents of the specific resin E-1 and the mixed solution C-1 in the obtained composition and the pH of the composition are shown in Table 1 later.

Regarding the type and amount of the pH adjusting agent, the pH of the mixed solution of the specific resin E-1 and the mixed solution C-1 was measured, and in a case where the pH of the mixed solution was larger than the value shown in Table 1, citric acid was added; and in a case where the pH of the mixed solution was smaller than the value shown in Table 1, monoethanolamine was added in an amount such that the pH of the mixed solution was the value shown in Table 1.

According to the preparation method for the composition of Example A1, each of compositions of Examples and Comparative Examples, which had formulations shown in Tables 1 and 2 later, was prepared.

### Evaluation of Composition

The following tests were carried out using each of the prepared compositions.

### <Solubility>

Each of substrates (Si) on which a W film consisting of a tungsten simple body (W) and a TiAlC film consisting of titanium-aluminum carbide (TiAlC) were respectively laminated at a film thickness of 100 nm was produced, and each of these substrates was cut into a square shape of 2 cm×2 cm to prepare a specimen.

Each of the obtained specimens was immersed in each composition (liquid temperature: 80° C.) for 10 minutes.

Before and after the above-described immersion test, the film thickness of each film was measured with a fluorescent X-ray analysis device (XRF AZX-400, manufactured by Rigaku Corporation) for thin film evaluation. From the measured film thicknesses before and after immersion, the dissolution rate (Å/min) of each film in a case where each composition was used was calculated. From the calculated dissolution rate, the solubility for the W film and the TiAlC film was evaluated based on the following evaluation standard.

The evaluation results of the solubility for the obtained W film and TiAlC film are shown in Table 1 later. As the dissolution rate is slower, the dissolution of each film was further suppressed and indicated by “S” as the highest evaluation of solubility.

### (Evaluation Standard for Solubility of W Film)

S: dissolution rate of the W film was 0.5 Å/min or less.
A: dissolution rate of the W film was more than 0.5 Å/min and 0.7 Å/min or less.
B: dissolution rate of the W film was more than 0.7 Å/min and 1.0 Å/min or less.
C: dissolution rate of the W film was more than 1.0 Å/min and 3.0 Å/min or less.
D: dissolution rate of the W film was more than 3.0 Å/min and 5.0 Å/min or less.
E: dissolution rate of the W film was more than 5.0 Å/min.

### (Evaluation Standard for Solubility of TiAlC Film)

S: dissolution rate of the TiAlC film was 0.5 Å/min or less.
A: dissolution rate of the TiAlC film was more than 0.5 Å/min and 0.7 Å/min or less.
B: dissolution rate of the TiAlC film was more than 0.7 Å/min and 1.0 Å/min or less.
C: dissolution rate of the TiAlC film was more than 1.2 Å/min and 1.5 Å/min or less.
D: dissolution rate of the TiAlC film was more than 1.5 Å/min and 1.7 Å/min or less.
E: dissolution rate of the TiAlC film was more than 1.7 Å/min.

### <Residue Removability>

A laminate (corresponding to a laminate before treatment) including a W film, an SiO₂ film, and a metal hard mask (TiN) having a predetermined opening portion in this order was formed on a substrate (Si). Using the obtained laminate, dry etching was carried out using the metal hard mask as a mask, and the SiO₂ film was etched until the surface of the W film was exposed to form holes, thereby preparing a sample 1 (see FIG. 1). As a result of confirming a cross section of the laminate with a photographic image from a scanning electron microscope (SEM), dry etching residues were observed on the wall surface in the holes.

The removability (residue removability) of the dry etching residues was evaluated according to the following procedure.

First, a section (having a square shape of approximately 2.0 cm× 2.0 cm) of the prepared sample 1 was immersed in each composition in which the temperature was adjusted to 80° C. Immediately after 3 minutes had elapsed from the start of the immersion, a section of the sample 1 was taken out, immediately washed with ultrapure water, and dried with N₂. Thereafter, a surface of the section of the immersed sample 1 was observed with SEM to confirm the presence or absence of dry etching residues.

Similarly, the section of the sample 1, which had been immersed for 5 minutes, was washed with ultrapure water and dried with N₂, and then a surface of the section was observed with SEM to confirm the presence or absence of dry etching residues.

The residue removability was evaluated according to the following determination standard from the observation results of each of sections of the sample 1.

### (Evaluation Standard for Residue Removability)

A: dry etching residues were completely removed by immersion for 3 minutes.
B: dry etching residues were not completely removed by immersion for 3 minutes, but were completely removed by immersion for 5 minutes.
C: dry etching residues were completely removed by immersion for 5 minutes.
   <Evaluation of Residual Properties of Composition after Rinsing Treatment>

A substrate (Si) including an SiO₂ film was produced, and the substrate (Si) including the SiO₂ film was subjected to a treatment of immersing for 10 minutes in each composition adjusted to 80° C. Next, the substrate subjected to the immersion treatment was immersed in a rinsing liquid consisting of isopropanol for 0.5 minutes to carry out a rinsing treatment of the SiO₂ film.

The surface of the SiO₂ film subjected to the rinsing treatment was analyzed by X-ray photoelectron spectroscopy, and a ratio (unit: atom %) of the number of nitrogen atoms derived from each composition (particularly, the specific resin) to the number of all atoms on the surface of the SiO₂ film was measured.

The measurement conditions of the X-ray photoelectron spectroscopy are shown below.

### (Measurement Conditions)

Device: Quantera SXMTM manufactured by ULVAC-PHI. Inc.
X-ray source: Monochromatic A1 Kα ray
X-ray beam diameter: φ200 μm
Signal capture angle: 45°

From the measurement results of the number ratio of nitrogen atoms to all atoms on the surface of the SiO₂ film, residual properties (residual properties after rinsing) of the composition after the rinsing treatment were evaluated.

The obtained evaluation results are shown in Table 1 later.

It is preferable that the above-described number ratio of nitrogen atoms to all atoms is smaller. The smaller the number ratio of nitrogen atoms to all atoms is, the higher the solubility of each component (particularly, the specific resin) of the composition in the rinsing liquid is, which means the residual amount of the composition on the surface of the SiO₂ film after the rinsing treatment is small.

(Evaluation Standard for Residual Properties after Rinsing)
A: number ratio of nitrogen atoms to all atoms on the surface of the SiO₂ film was 0.1 atom % or less.
B: number ratio of nitrogen atoms to all atoms on the surface of the SiO₂ film was more than 0.1 atom % and 1.0 atom % or less.
C: number ratio of nitrogen atoms to all atoms on the surface of the SiO₂ film was more than 1.0 atom % and 3.0 atom % or less.
D: number ratio of nitrogen atoms to all atoms on the surface of the SiO₂ film was more than 3.0 atom %.

Table 1 and Table 2 show the formulation of each composition used in each of Examples and each of Comparative Examples, and Table 1 shows the evaluation results of each of Examples and each of Comparative Examples.

Table 2 shows the formulation of the components of the mixed solutions C-1 to C-6 used for preparing the above-described compositions.

Table 1 shows the content of the resin and the mixed solution contained in each composition and the pH of each composition, and Table 2 shows the type and content of each component contained in each mixed solution.

In Table 1, the column of “Ratio a/b” of “Specific resin” indicates a ratio a/b of a molar number a of the repeating unit A to a molar number b of the repeating unit B in the specific resin having the repeating unit A and the repeating unit B.

In Table 1, the column of “Amount (part by mass)” of “Specific resin” indicates the content of the specific resin in a case where the total mass of the composition is set to 100 parts by mass.

In Table 1, the column of “pKa” of “Specific resin” indicates the pKa of the functional group having a pKa of 10.0 or less, in the repeating unit B.

In Table 1, the column of “Amount (part by mass)” of “Mixed solution” indicates the content of the mixed solution in a case where the total mass of the composition is set to 100 parts by mass.

In Table 1, the column of “pH” indicates the pH of the above-described composition at 25° C. which was measured with the pH meter.

In Table 2, the numerical values shown in each column mean the content (unit: part by mass) of each component having a numerical value in a case where the total mass of each mixture is set to 100 parts by mass.

TABLE 1

| | | | Example | Example | Example | Example | Example | Example | Example |
|---|---|---|---|---|---|---|---|---|---|
| Table 1 | | | A1 | A2 | A3 | A4 | A5 | A6 | A7 |
| Composition | Specific | Type | E-1 | E-2 | E-3 | E-4 | E-5 | E-6 | E-7 |
| | resin | Ratio a/b | 50/50 | 50/50 | 50/50 | 50/50 | 50/50 | 50/50 | 25/25/50 |
| | | Amount (part | 0.05 | 0.05 | 0.05 | 0.05 | 0.05 | 0.05 | 0.05 |
| | | by mass) | | | | | | | |
| | | pKa | 4 | 4 | 4 | 4 | 4 | 4 | 4 |
| | Mixed | Type | C-1 | C-1 | C-1 | C-1 | C-1 | C-1 | C-1 |
| | solution | Amount (part | 99.95 | 99.95 | 99.95 | 99.95 | 99.95 | 99.95 | 99.95 |
| | | by mass) | | | | | | | |
| | pH | | 9 | 9 | 9 | 9 | 9 | 9 | 9 |
| Evaluation | Solubility | W | S | A | B | B | S | B | B |
| | | TiAlC | S | B | A | A | A | A | B |

| | | | | | | | |
|---|---|---|---|---|---|---|---|
| Residue removability | A | B | B | B | A | B | B |
| Residual properties | A | A | A | A | A | A | A |
| after rinsing | | | | | | | |

| | | | Example | Example | Example | Example | Example | Example |
|---|---|---|---|---|---|---|---|---|
| Table 1 | | | A8 | A9 | A10 | A11 | A12 | A13 |
| Composition | Specific | Type | E-8 | E-9 | E-10 | E-11 | E-12 | E-13 |
| | resin | Ratio a/b | 50/50 | 50/50 | 50/50 | 50/50 | 50/50 | 50/50 |
| | | Amount (part | 0.05 | 0.05 | 0.05 | 0.05 | 0.05 | 0.05 |
| | | by mass) | | | | | | |
| | | pKa | 4 | 10 | 4 | 10 | 10 | 10 |
| | Mixed | Type | C-1 | C-1 | C-1 | C-1 | C-1 | C-1 |
| | solution | Amount (part | 99.95 | 99.95 | 99.95 | 99.95 | 99.95 | 99.95 |
| | | by mass) | | | | | | |
| | pH | | 9 | 9 | 9 | 9 | 9 | 10 |
| Evaluation | Solubility | W | B | B | A | A | S | C |
| | | TiAlC | C | C | A | B | A | C |

| | | | | | | |
|---|---|---|---|---|---|---|
| Residue removability | B | B | B | B | A | B |
| Residual properties | A | B | A | B | B | B |
| after rinsing | | | | | | |

TABLE 2

| | | | Example | Example | Example | Example | Example | Example |
|---|---|---|---|---|---|---|---|---|
| Table 1 | | | A14 | A15 | A16 | A17 | A18 | A19 |
| Composition | Specific | Type | E-14 | E-15 | E-16 | E-17 | E-18 | E-19 |
| | resin | Ratio a/b | 50/50 | 50/50 | 50/50 | 50/50 | 50/50 | 5/95 |
| | | Amount (part | 0.05 | 0.05 | 0.05 | 0.05 | 0.05 | 0.05 |
| | | by mass) | | | | | | |
| | | pKa | 4 | 4 | 4 | −2 | 4 | 4 |
| | Mixed | Type | C-2 | C-3 | C-4 | C-5 | C-6 | C-1 |
| | solution | Amount (part | 99.95 | 99.95 | 99.95 | 99.95 | 99.95 | 99.95 |
| | | by mass) | | | | | | |
| | pH | | 10 | 9 | 5 | 4 | 10 | 9 |
| Evaluation | Solubility | W | S | S | S | S | S | S |
| | | TiAlC | S | S | S | S | S | S |

| | | | | | | |
|---|---|---|---|---|---|---|
| Residue removability | A | A | A | A | A | A |
| Residual properties | A | A | A | A | A | A |
| after rinsing | | | | | | |

| | | | | | Comparative | Comparative |
|---|---|---|---|---|---|---|
| | | | Example | Example | Example | Example |
| Table 1 | | | A20 | A21 | A1 | A2 |
| Composition | Specific | Type | E-1 | E-1 | CE-1 | CE-2 |
| | resin | Ratio a/b | 50/50 | 50/50 | — | 30/20/50 |
| | | Amount (part | 0.001 | 1.005 | 0.05 | 0.05 |
| | | by mass) | | | | |
| | | pKa | 4 | 4 | — | 4 |
| | Mixed | Type | C-1 | C-1 | C-1 | C-1 |
| | solution | Amount (part | 99.999 | 99.00 | 99.95 | 99.95 |
| | | by mass) | | | | |
| | pH | | 9 | 9 | 9 | 10 |
| Evaluation | Solubility | W | S | S | E | D |
| | | TiAlC | S | S | E | D |

| | | | | |
|---|---|---|---|---|
| Residue removability | A | A | C | C |
| Residual properties | A | A | D | C |
| after rinsing | | | | |

TABLE 3
[Table 2]

| Mixed solution | | C-1 | C-2 | C-3 | C-4 | C-5 | C-6 |
|---|---|---|---|---|---|---|---|
| Removing | Hydroxylamine | 5.00 | | 4.50 | | | |
| agent | Ammonium hydroxide | | 5.00 | | | | |
| | Tetramethylammonium hydroxide | | | 0.50 | | | |
| | Hydrofluoric acid | | | | 5.00 | | |
| | Sulfuric acid | | | | | 2.50 | |
| Oxidizing | Hydrogen peroxide | | | | | 2.50 | |
| agent | | | | | | | |
| Corrosion | 5-Methyl-1H-benzotriazole | 0.06 | 0.06 | | | 0.06 | |
| inhibitor | 5-Mercapto-1-phenyl-1H-tetrazole | | | 0.06 | | | |
| | n-Dodecyl mercaptan | | | | 0.06 | | |
| Surfactant | PERSOFT SF-T | 1.00 | | | | | |
| | NISSAN CATION BB | | 1.00 | | | | |
| | NONION K-220 | | | | 1.00 | | |
| | NISSAN ANON BL | | | | | 1.00 | |
| Antifoaming | KF-6701 | | 1.00 | | | | |
| agent | | | | | | | |
| Solvent | Propylene glycol | | | 4.00 | | | |
| | Water | 93.94 | 92.94 | 90.94 | 93.94 | 93.94 | 100.00 |

| | | | | | | |
|---|---|---|---|---|---|---|
| Total (part by mass) | 100.00 | 100.00 | 100.00 | 100.00 | 100.00 | 100.00 |

From the results of Table 1, it was found that the present composition had excellent solubility of the W film, excellent solubility of the TiAlC film, excellent removability for etching residues, and excellent residual properties of the composition after the rinsing treatment.

On the other hand, the compositions of Comparative Examples included a resin having a repeating unit having a pyrrolidone skeleton or a resin having a repeating unit having the specific group and a repeating unit having a hydroxy group, but no sufficient effect was obtained in any of the solubility of the W film, the solubility of the TiAlC film, the removability for etching residues, and the residual properties of the composition after the rinsing treatment.

In addition, from the results of Table 1, regarding the specific resin, it was found that, in a case where the repeating unit A was the repeating unit derived from the compound represented by Formula (A), the repeating unit derived from the compound represented by Formula (B), or the repeating unit represented by Formula (C), the solubility of the W film was more excellent (comparison between Example A9 and Example A13, and the like).

Regarding the specific resin, it was found that, in a case where at least one of the specific groups was a primary amino group or a salt thereof, the solubility of the TiAlC film was more excellent (comparison of Examples A6 to A8, and the like).

Regarding the specific resin, it was found that, in a case where the repeating unit A was the repeating unit derived from the compound represented by Formula (A), in which L^{a} was an (n+2)-valent linking group including an aromatic ring, the repeating unit derived from the compound represented by Formula (B), in which at least one of L^{b1} or L^{b2} was a divalent linking group including an aromatic ring, or the repeating unit represented by Formula (C), the solubility of the W film was more excellent (comparison between Examples A6 and A10, and the like).

Regarding the specific resin, it was found that, in a case where the repeating unit A was the repeating unit derived from the compound represented by Formula (D) or the repeating unit represented by Formula (E), the solubility of the W film was more excellent (comparison between Examples A1 and A10, and the like).

Regarding the specific resin, it was found that, in a case where the repeating unit B had a functional group having a pKa of 5.0 or less, the residual properties of the composition after the rinsing treatment were more excellent (comparison between Examples A1 and A12, and the like).

It was found that, even in a case where the specific resin E-14 and the specific resin E-18 were used together at 0.025 parts each instead of the specific resin E-1 used in Example A1 in Table 1, and the specific resin E-14 and the specific resin E-18 were used together at 0.05 parts in total, the same evaluation results as those of the composition described in Example A1 were obtained in the evaluations described above.

In addition, it was found that the same evaluation results as in Example A1 were exhibited even in a case where the specific resin E-15 and polymethacrylic acid were used together in an amount of 0.025 parts each instead of the specific resin E-1 used in Example A1, and the specific resin E-15 and polymethacrylic acid were used together in an amount of 0.05 parts in total, and the evaluations described above were carried out.

### Example B

### Preparation of Composition

A preparation method for each of the compositions of Examples B1 to B5 and Comparative Examples B1 and B2 will be described with reference to Example B1.

The specific resin E-1, citric acid, trishydroxymethylaminomethane (Tris), and ultrapure water were mixed such that amounts thereof were the contents shown in Table 3 later. Thereafter, potassium hydroxide or nitric acid as a pH adjusting agent was added thereto such that the pH of the composition to be prepared was 6.0, and the obtained mixed liquid was sufficiently stirred with a stirrer to prepare a composition of Example B1.

According to the preparation method for the composition of Example B1, each of compositions of Examples and Comparative Examples, which had formulations shown in Table 3 later, was prepared.

### [Evaluation of Composition]

### <Residue Removability>

Using each of the prepared compositions, residue removability (washing performance) was evaluated in a case where a metal film subjected to the CMP treatment was washed.

Specifically, using FREX-300SII (a polishing device, manufactured by Ebara Corporation), a wafer (diameter: 12 inches) having a metal film consisting of tungsten on a surface was subjected to a CMP treatment using a polishing liquid (W2000, manufactured by Cabot Corporation) under conditions of a polishing liquid supply rate of 0.28 mL/(min·cm²), a polishing pressure of 2.0 psi, and a polishing time of 60 seconds.

Thereafter, the temperature of each composition was adjusted to room temperature (23° C.), and scrub washing was carried out using each composition for 60 seconds, followed by drying. The number of defects on the polished surface of the obtained wafer was detected using a defect detection device, and each defect was observed with a scanning electron microscope (SEM) and subjected to defect classification. In a case of being necessary, the constitutional elements were analyzed by energy dispersion type X-ray analysis apparatus (EDAX) to specify the components. In this manner, the number of defects based on the residues due to the CMP treatment was determined, and the washing performance was evaluated according to the following evaluation standard (an evaluation 6 indicates that the washing performance was most excellent).

### (Evaluation Standard for Residue Removability)

6: number of target defects was less than 20.
5: number of target defects was 20 or more and less than 50.
4: number of target defects was 50 or more and less than 100.
3: number of target defects was 100 or more and less than 200.
2: number of target defects was 200 or more and less than 300.
1: number of target defects was 300 or more.

### <Solubility>

Using each composition, the solubility for the metal film was evaluated in a case where the metal film was washed.

Specifically, a wafer (diameter: 12 inches) having a W film consisting of a tungsten simple body (W) on the surface was cut to prepare a 2 cm×2 cm square wafer coupon. A thickness of the W film was set to 200 nm. The wafer coupon was immersed in each composition (liquid temperature: 23° C.) for 30 minutes under a stirring condition of a stirring rotation speed of 250 rpm.

Before and after the above-described immersion test, the film thickness of the W film was measured with an optical film thickness meter Ellipsometer M-2000 (manufactured by J.A. Woollam Co.). From the measured film thicknesses before and after immersion, the dissolution rate (A/min) of the W film in a case where each composition was used was calculated. From the calculated dissolution rate, the solubility of the W film (the performance of suppressing the dissolution of the W film) was evaluated based on the following evaluation standard. It is indicated that the slower the dissolution rate is, the more suppression of the dissolution of the W film occurs by the composition (an evaluation 6 indicates that the solubility was most excellent).

### (Evaluation Standard for Solubility of W Film)

6: dissolution rate of the W film was 0.5 Å/min or less.
5: dissolution rate of the W film was more than 0.5 Å/min and 0.7 Å/min or less.
4: dissolution rate of the W film was more than 0.7 Å/min and 1.0 Å/min or less.
3: dissolution rate of the W film was more than 1.0 Å/min and 3.0 Å/min or less.
2: dissolution rate of the W film was more than 3.0 Å/min and 5.0 Å/min or less.
1: dissolution rate of the W film was more than 5.0 Å/min.

Table 3 shows the formulation of each composition used in each of Examples and each of Comparative Examples, and the evaluation results thereof.

In Table 3, the column of “Amount %)” of each component indicates the content (unit: % by mass) of each component with respect to the total mass of the composition.

In Table 3, the numerical value in the column of “Organic acid/amine” indicates the content of the organic acid (removing agent) with respect to the content of the basic compound (removing agent).

In Table 3, “Remainder” in the column of “Water” indicates that the water content was the remainder of the composition other than the resin, the removing agent, and the pH adjusting agent.

In Table 3, the numerical value in the column of “pH” indicates the pH of the above-described composition at 25° C. which was measured with the pH meter.

TABLE 4

| | Composition | | | | | |
|---|---|---|---|---|---|---|
| | Specific resin | | Removing agent | | | |
| | | Amount | | Amount | | Amount |
| Table 3 | Type | (%) | Type | (%) | Type | (%) |
| Example B1 | E-1 | 0.05 | Citric | 1.5 | Trishydroxymethylaminomethane | 4.5 |
| | | | acid | | | |
| Example B2 | E-5 | 0.05 | Tartaric | 1 | TMAH | 2 |
| | | | acid | | | |
| Example B3 | E-8 | 0.05 | Succinic | 2 | Monoethanolamine | 3 |
| | | | acid | | | |
| Example B4 | E-13 | 0.05 | Citric | 1 | Tris(2-hydroxyethyl)methylammonium | 3.4 |
| | | | acid | | hydroxide | |
| Example B5 | E-16 | 0.05 | EDTA | 3 | 2-Dimethylamino-2-methyl-1-propanol | 4.2 |
| Comparative | — | — | Citric | 1.5 | Trishydroxymethylaminomethane | 4 |
| Example B1 | | | acid | | | |
| Comparative | CE-1 | 0.05 | Citric | 1.5 | Trishydroxymethylaminomethane | 3.5 |
| Example B2 | | | acid | | | |

| | Composition | | | | Evaluation | |
|---|---|---|---|---|---|---|
| | | | pH | | | Solubility |
| | Organic | | adjusting | | Residue | for W |
| Table 3 | acid/amine | Water | agent | pH | removability | film |
| Example B1 | 0.3 | Remainder | KOH/HNO₃ | 6.0 | 6 | 6 |
| Example B2 | 0.5 | Remainder | KOH/HNO₃ | 5.0 | 6 | 6 |
| Example B3 | 0.7 | Remainder | KOH/HNO₃ | 6.5 | 5 | 4 |
| Example B4 | 0.3 | Remainder | KOH/HNO₃ | 9.0 | 5 | 3 |
| Example B5 | 0.7 | Remainder | KOH/HNO₃ | 4.0 | 6 | 6 |
| Comparative | 0.4 | Remainder | KOH/HNO₃ | 6.0 | 6 | 1 |
| Example B1 | | | | | | |
| Comparative | 0.4 | Remainder | KOH/HNO₃ | 7.0 | 6 | 2 |
| Example B2 | | | | | | |

From the results of Table 3, it was found that, in the compositions of Examples according to the present invention, both the solubility of the W film and the removability for residues on the wafer subjected to the CMP treatment were excellent. On the other hand, the compositions of Comparative Examples, which did not contain the specific resin, had a low evaluation of the solubility of the W film, and thus the above-described effect was not obtained.

In the evaluation test of the residue removability, a wafer having a metal film consisting of W on a surface was subjected to a CMP treatment, and then the polished surface of the wafer subjected to the CMP treatment was subjected to a buff polishing treatment.

In the buff polishing treatment, each composition adjusted to room temperature (23° C.) was used as a washing solution for buff polishing. In addition, a buff polishing treatment was carried out using the polishing device used in the CMP treatment under conditions of a polishing pressure of 2.0 psi, a supply rate of the washing solution for buff polishing of 0.28 mL/(min·cm²), and a polishing time of 60 seconds.

Thereafter, the wafer which had been subjected to the buff polishing treatment was washed over 30 seconds using each composition adjusted to room temperature (23° C.) and then subjected to a drying treatment.

As a result of evaluating the residue removability of the composition with respect to the obtained polished surface of the wafer according to the test method described in <Residue removability> described above, it was found that the same evaluation results as those of the composition of each of Examples described above were exhibited.

### EXPLANATION OF REFERENCES

1: substrate
2: metal layer
3: etching stop layer
4: insulating film
5: metal hard mask
6: hole
10: laminate
11: interior wall
11a: cross-sectional wall
11b: bottom wall
12: dry etching residue

What is claimed is:

1. A composition for a semiconductor device, comprising:
a resin including a repeating unit A which has a specific group selected from the group consisting of a primary amino group, a secondary amino group, a tertiary amino group, and salts of these groups, and has a hydroxy group, and
a repeating unit B which has a functional group having a pKa of 10.0 or less or a salt of the functional group; and
water.

2. The composition for a semiconductor device according to claim 1,
wherein the repeating unit A is a repeating unit derived from a compound represented by Formula (A), a repeating unit derived from a compound represented by Formula (B), or a repeating unit represented by Formula (C), 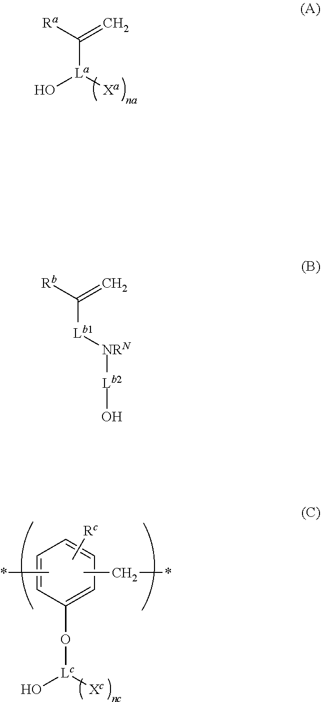
in Formula (A),
R^{a} represents a hydrogen atom or an alkyl group having 1 to 4 carbon atoms,
X^{a} represents a specific group selected from the group consisting of a primary amino group, a secondary amino group, a tertiary amino group, and salts of these groups,
L^{a} represents an (na+2)-valent linking group, and
na represents an integer of 1 to 5,
in Formula (B),
R^{b} represents a hydrogen atom or an alkyl group having 1 to 4 carbon atoms,
R^{N} represents a hydrogen atom or an alkyl group which may have a substituent, and
L^{b1} and L^{b2} each independently represent a single bond or a divalent linking group,
in Formula (C),
* represents a bonding position,
R^{c} represents a hydrogen atom or a methyl group,
X^{c} represents a specific group selected from the group consisting of a primary amino group, a secondary amino group, a tertiary amino group, and salts of these groups,
L^{c} represents an (nc+2)-valent linking group, and
nc represents an integer of 1 to 5.

3. The composition for a semiconductor device according to claim 1,
wherein at least one of the specific group is a primary amino group or a salt of the primary amino group.

4. The composition for a semiconductor device according to claim 2,
wherein the repeating unit A is the repeating unit derived from the compound represented by Formula (A), in which L^{a} is the (na+2)-valent linking group including an aromatic ring, the repeating unit derived from the compound represented by Formula (B), in which at least one of L^{b1} or L^{b2} is a divalent linking group including an aromatic ring, or the repeating unit represented by Formula (C).

5. The composition for a semiconductor device according to claim 1,
wherein the repeating unit A is a repeating unit derived from a compound represented by Formula (D) or a repeating unit represented by Formula (E), 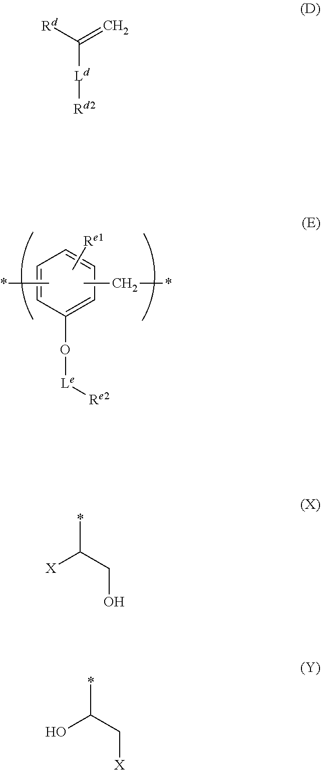
in Formula (D),
R^{d} represents a hydrogen atom or an alkyl group having 1 to 4 carbon atoms,
L^{d} represents a single bond or a divalent linking group, and
R^{d2} represents a group represented by Formula (X) or a group represented by Formula (Y),
where in Formula (X) and Formula (Y), * represents a bonding position, and
X represents a primary amino group or a salt of the primary amino group,
in Formula (E),
* represents a bonding position,
R^{e1} represents a hydrogen atom or a methyl group,
L^{e} represents a divalent linking group, and
R^{e2} represents the group represented by Formula (X) or the group represented by Formula (Y).

6. The composition for a semiconductor device according to claim 1,
wherein the pKa of the functional group is 5.0 or less.

7. The composition for a semiconductor device according to claim 1,
wherein the functional group is a carboxy group.

8. The composition for a semiconductor device according to claim 1, further comprising:
at least one selected from the group consisting of a removing agent, an oxidizing agent, a corrosion inhibitor, a surfactant, an antifoaming agent, and an organic solvent.

9. The composition for a semiconductor device according to claim 1, further comprising:
a removing agent.

10. A substrate treatment method comprising:
a step A of bringing the composition for a semiconductor device according to claim 1 into contact with a substrate having a metal-containing substance.

11. A substrate treatment method comprising:
a step A of bringing the composition for a semiconductor device according to claim 2 into contact with a substrate having a metal-containing substance.

12. A substrate treatment method comprising:
a step A of bringing the composition for a semiconductor device according to claim 3 into contact with a substrate having a metal-containing substance.

13. A substrate treatment method comprising:
a step A of bringing the composition for a semiconductor device according to claim 4 into contact with a substrate having a metal-containing substance.

14. A substrate treatment method comprising:
a step A of bringing the composition for a semiconductor device according to claim 5 into contact with a substrate having a metal-containing substance.

15. A substrate treatment method comprising:
a step A of bringing the composition for a semiconductor device according to claim 6 into contact with a substrate having a metal-containing substance.

16. A substrate treatment method comprising:
a step A of bringing the composition for a semiconductor device according to claim 7 into contact with a substrate having a metal-containing substance.

17. A substrate treatment method comprising:
a step A of bringing the composition for a semiconductor device according to claim 8 into contact with a substrate having a metal-containing substance.

18. The substrate treatment method according to claim 10, further comprising, after the step A:
a step B of subjecting the substrate obtained in the step A to a rinsing treatment using a rinsing liquid.

19. A manufacturing method of a semiconductor device, comprising:
the substrate treatment method according to claim 10.

20. A compound represented by Formula (F), 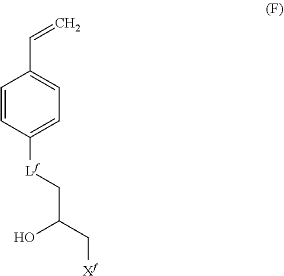
in Formula (F),
X^{f} represents a specific group selected from the group consisting of a primary amino group, a secondary amino group, a tertiary amino group, and salts of these groups, and
L^{f} represents —CH₂O—, —COO—, —C≡C—CH₂O—, or —O—.
